(19) United States
(12) Patent Application Publication
(10) Pub. No.: US 2007/0167466 A1
(43) Pub. Date: Jul. 19, 2007

(54) "Pyrazolo [4,3-d]pyrimidines, processes for their preparation and methods of use"

(76) Inventors: Daniela Moravcova, Horazdovice (CZ); Libor Havlicek, Praha (CZ); Vladimir Krystof, Ostrava (CZ); Rene Lenobel, Frydek Mistek (CZ); Pavla Binarova, Olomouc (CZ); Petr Mlejnek, Brno (CZ); Borek Vojtesek, Modrice (CZ); Stjepan Uldrijan, Brno (CZ); Thomas Schmulling, Berlin (DE); Miroslav Strnad, Olomouc (CZ)

Correspondence Address: MICHAEL BEST & FRIEDRICH, LLP, 100 E WISCONSIN AVENUE, Suite 3300, MILWAUKEE, WI, 53202, US

(21) Appl. No.: 10/555,528

(22) PCT Filed: May 6, 2004
(86) PCT No.: PCT/EP04/04855

§ 371 Date: Aug. 3, 2006

(30) Foreign Application Priority Data
May 6, 2003 (EP) 030 10 184.4

(51) Int. Cl. A61K 31/519; C07D 487/02

(52) U.S. Cl. 514/262.1; 544/262

(57) ABSTRACT

The invention relates to 3-,7-disubstituted pyrazolo[4,3-d]pyrimidines represented by the general formula I (I), and pharmaceutically acceptable salts thereof, wherein R3 is selected from the group consisting of alkyl, cycloalkyl, cycloalkyl alkyl, cycloheteroalkyl alkyl, cycloheteroalkyl, aryl, heterocycle, heteroaryl, arylalkyl, heteroarylalkyl, and heteroalkyl, wherein each of the groups may optionally be substituted, R7 is selected from the group consisting of halogen, hydroxyl, hydroxylamino, amino, carboxyl, cyano, nitro, amido, sulfo, sulfamido, carbamino, unsubstituted or substituted alkyl, substituted or unsubstituted cycloalkyl, substituted or unsubstituted arylalkyl, substituted or unsubstituted heteroalkyl, substituted or unsubstituted heteroarylalkyl, substituted or unsubstituted cycloalkyl alkyl, substituted or unsubstituted cycloheteroalkyl alkyl; R7′-X— wherein X is an —NH—, —N(alkyl)-, -0- or —S— moiety and R7′ is selected from the group consisting of H, alkyl, cycloalkyl, aryl, alkylcycloalkyl, arylalkyl, heterocycle, heterocyclealkyl, substituted alkyl, substituted cycloalkyl, substituted aryl, substituted arylalkyl, substituted heterocycle, substituted heteroaryl, substituted heteroarylalkyl, substituted heteroalkyl, substituted cycloalkyl alkyl and substituted cycloheteroalkyl alkyl, wherein the groups are preferably substituted by more than one halogen, hydroxyl, amino, mercapto, carboxyl, cyano, nitro, amido, sulfo, sulfamido, carbamino, alkyl, alkoxy, and substituted alkyl group.

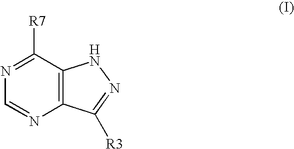

This invention relates to new pyrazolo[4,3-d]pyrimidine derivatives and to their use in suitable utilities, especially in cancer therapy and agricultural practice.

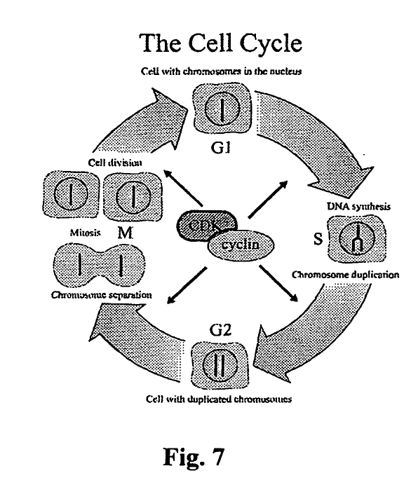

The cell division cycle is an evolutionarily conserved process in all eukaryotic cells to control growth and division. The cell cycle consists of four distinct stages illustrated in FIG. 7, the G1, S, G2 and M phase. Normal cellular proliferation is initiated and tightly controlled by a series of regulatory mechanisms that either permit or prevent cell cycle progression. In every phase, there are protein complexes, the cyclins and cyclin-dependent kinases regulating and advancing the cell cycle. FIG. 7 shows the cell division cycle (cdc) consisting of four phases G1, S, G2 and M. Mitosis (the actual division) occurs in M-phase. In every phase, there are specific cyclin-dependent kinase complexes present (CDK's).

Proliferative disorders such as cancer are recognised as diseases of the cell cycle. It has been found that in tumour cells, the mechanisms that normally function to restrain cell division are defective, whilst those that promote division become more active. The genes responsible for these changes in growth and proliferation are generally named “tumour suppressors” and “oncogenes”. Cell-cycle regulatory compounds are pivotal in the modulation of abnormal cellular proliferation as they provide ideal targets for therapy for a range of proliferative disorders.

A series of 3-,7-disubstituted pyrazolo[4,3-d]pyrimidines are useful for inhibition of cyclin-dependent kinases (preferably p34^{cdc2}/cyclin B). Hence they can be used as antimitotic and apoptotic drugs, particularly as anticancer drugs and herbicides. Likewise, the compounds can be used as anti-fungal agents, which may have high value in the treatment of aspergillosis, penicilliosis, actinomycosis and the like. Difference in homology of insect CDK genes permit selection of compounds of this invention which discriminate between insect/mammalian CDK enzymes and thus leads to insecticides.

### SUMMARY OF THE INVENTION

It is an object of this invention to provide antimitotic, anticancer, herbicidal, fungicidal and insecticidal compounds having improved selectivity and efficiency index, i.e. that are less toxic yet more efficacious than analogues known heretofore.

It is an object of this invention to provide 3,7-disubstituted pyrazolo[4,3-d]pyrimidines, which inhibit the cdks, cell proliferation or block cytokinin receptors.

A further object of this invention is to provide a pharmaceutical composition, which comprises a 3,7-disubstituted pyrazolo[4,3-d]pyrimidine, and a pharmaceutically acceptable carrier.

A further object of this invention to provide a method for inhibiting cell proliferation and/or inducing apoptosis to a mammal or plant in need of an effective amount of 3,7-disubstituted pyrazolo[4,3-d]pyrimidines.

This invention further constitutes a method for inhibiting cell proliferation to a plant in need of an effective amount 3,7-disubstituted pyrazolo[4,3-d]pyrimidines.

The solution of this object are 3-,7-disubstituted pyrazolo[4,3-d]pyrimidines represented by the general formula I 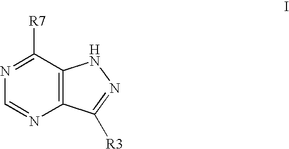
and pharmaceutically acceptable salts thereof, wherein
R3 is selected from the group consisting of
alkyl, cycloalkyl, cycloalkyl alkyl, cycloheteroalkyl alkyl, cycloheteroalkyl, aryl, heterocycle, heteroaryl, arylalkyl, heteroarylalkyl, and heteroalkyl, wherein each of the groups may optionally be substituted,
R7 is selected from the group consisting of halogen, hydroxyl, hydroxylamino, amino, carboxyl, cyano, nitro, amido, sulfo, sulfamido, carbamino, unsubstituted or substituted alkyl, substituted or unsubstituted cycloalkyl, substituted or unsubstituted arylalkyl, substituted or unsubstituted heteroalkyl, substituted or unsubstituted heteroarylalkyl, substituted or unsubstituted cycloalkyl alkyl, substituted or unsubstituted cycloheteroalkyl alkyl;
R7′-X— wherein X is an —NH—, —N(alkyl)-, —O— or —S— moiety and
R7′ is selected from the group consisting of H, alkyl, cycloalkyl, aryl, alkylcycloalkyl, arylalkyl, heterocycle, heterocycloalkyl, substituted alkyl, substituted cycloalkyl, substituted aryl, substituted arylalkyl, substituted heterocycle, substituted heteroaryl, substituted heteroarylalkyl, substituted heteroalkyl, substituted cycloalkyl alkyl and substituted cycloheteroalkyl alkyl.
If the above groups are substituted, they are preferably substituted by halogen, hydroxyl, amino, mercapto, carboxyl, cyano, nitro, amido, sulfo, sulfamido, carbamino, alkyl, alkoxy, and/or substituted alkyl group, in particular by more than one of the above substituents, preferably by 1 to 3 substituents.

In another embodiment, this invention is a method for inhibiting cdks and cell proliferation and/or for inducing apoptosis in plants, comprising administering an effective amount of a composition comprising one or more compounds according to claim 1 to the plant. The cdk inhibiting molecules are useful for treating disorders, some of them involving cell proliferation, and thus are useful as herbicides.

In yet another embodiment, this invention is a pharmaceutical composition comprising one or more compounds according to claim 1 in an admixture with one or more pharmaceutical excipients.

In still another embodiment, this invention is a composition comprising one or more compounds according to claim 1 useful for treating fungal infections (fungi) in plants.

In another embodiment, this invention is a composition comprising one or more compounds according to claim 1 useful for treating insects and yeasts on plants.

3,7-disubstituted pyrazolo[4,3-d]pyrimidines result in the acquisition of extremely high potency against plant viruses on the part of the defined compounds. As used herein, and unless modified by the immediate context:

“Halogen” preferably refers to fluorine, bromine, chlorine and iodine atoms.

“Hydroxy” refers to the group —OH.

“Mercapto” refers to group —SH.

“Alkyl” preferably refers to branched or unbranched C₁-C₈ alkyl chain which is saturated or unsaturated. Thus, the term “alkyl” when used herein encompasses alkyl alkenyl and alkinyl groups. Alkenyl groups preferably have 2 to 8 carbon atoms, alkinyl groups preferably have 3 to 8 carbon atoms. Such groups as methyl, ethyl, propyl, isopropyl, n-butyl, isobutyl, tert-butyl, allyl, ethinyl, propargyl, and the like can exemplify this term.

“Substituted alkyl” preferably refers to alkyl as described above including one to six, in particular 1 to 3 substituents such as hydroxyl, mercapto, alkylthio, halogen, alkoxy, acyloxy, amino, acylamino, hydrazino, carbamoyl, amido, carboxyl, sulfo, acyl, guanidino and the like. These groups may be attached to any carbon atom of the alkyl moiety.

“Alkoxy” denotes the group —OR, where R is preferably alkyl, substituted alkyl, aryl, substituted aryl, arylalkyl, substituted arylalkyl, cycloalkyl, substituted cycloalkyl, cycloheteroalkyl or substituted cycloheteroalkyl as defined herein.

“Alkylthio” denotes the group —SR, where R is preferably as defined for “alkoxy” group.

“Sulfo” denotes the group —SO₃R, where R is preferably H, alkyl or substituted alkyl as defined above.

“Sulfamido” denotes to the group SO₂NRR″ where R and R″ is preferably H, alkyl or substituted alkyl as defined above.

“Acyl” denotes groups —C(O)R, where R preferably is alkyl, substituted alkyl, aryl, substituted aryl, arylalkyl, substituted arylalkyl, cycloalkyl, substituted cycloalkyl as defined herein.

“Aryloxy” denotes groups —OAr, where Ar is preferably an aryl, substituted aryl, heteroaryl or substituted heteroaryl group as defined herein.

“Alkylamino” denotes the group —NRR′, where R and R′ may independently be hydrogen, alkyl, substituted alkyl, aryl, substituted aryl, heteroaryl or substituted heteroaryl as defined herein.

“Amido” denotes the group —C(O)NRR′, where R and R′ may independently be hydrogen, alkyl, substituted alkyl, aryl, substituted aryl, heteroaryl, substituted heteroaryl as defined herein.

“Carboxyl” denotes the group —C(O)OR, where R is preferably hydrogen, alkyl, substituted alkyl, aryl, substituted aryl, hetaryl or substituted hetaryl as defined herein.

“Acylamino denotes the group —NHCOR, where R may be alkyl, substituted alkyl, heterocycle, aryl, substituted aryl, heteroaryl and substituted heteroaryl as defined herein.

Carbamoylamino denotes the group NHCOOR, where R is preferably alkyl or aryl.

“Aryl” or “Ar” refers to an aromatic carbocyclic group having at least one aromatic ring (e.g., phenyl or biphenyl) or multiple condensed rings in which at least one ring is aromatic (e.g., 1,2,3,4tetrahydronaphthyl, naphthyl, anthryl, or phenanthryl).

Preferably, the aryl group has more than six, in particular 6 to 10 carbon atoms.

“Substituted aryl” refers to aryl as described above which is optionally substituted with one or more functional groups, in particular 1 to 3 substituents, such as halogen, alkyl, hydroxy, amino, acylamino, carbamoylamino, hydrazino, mercapto, alkoxy, alkylthio, alkylamino, amido, carboxyl, nitro, sulfo and the like as defined herein.

“Heterocycle” refers to a unsaturated or aromatic carbocyclic group preferably having 1 to 3 rings and having at least one, preferably 1 to 3 and in particular 1 or 2 hetero atoms, such as N, O or S, within the ring; the ring can be single (e.g. pyranyl, pyridyl or furyl) or multiple condensed (e.g., quinazolinyl, purinyl, quinolinyl or benzofuranyl) which can optionally be unsubstituted or substituted with, e.g., halogen, amino, hydroxy, cyano, nitro, mercapto, alkoxy, alkylamino, acylamino, carbamoylamino, acyloxy, dialkylamino, alkylthio carboxyl, amido, sulfo, sulfamido, and the like as defined above. The heterocycle groups preferably has 5 to 10 ring atoms, which are either carbon atoms or hetero atoms as defined above.

“Heteroaryl” refers to a heterocycle in which at least one heterocyclic ring is aromatic.

“Substituted heteroaryl” refers to a heterocycle optionally mono or poly substituted with one or more functional groups, preferably 1 to 6, in particular 1 to 3 substituents, e.g., halogen, amino, hydroxy, cyano, nitro, mercapto, alkoxy, alkylamino, acylamino, carbamoylamino, acyloxy, dialkylamino, alkylthio carboxyl, amido, sulfo, sulfamido, and the like.

“Arylalkyl” refers to the group —R—Ar where Ar is an aryl group and R is alkyl or substituted alkyl group as defined above. The aryl groups can optionally be unsubstituted or substituted as defined above with, e.g., halogen, amino, acylamino, carbamoylamino, hydrazino, acyloxy, alkyl, hydroxyl, alkoxy, alkylthio, alkylamino, amido, carboxyl, hydroxy, aryl, nitro, mercapto, sulfo and the like.

“Heteroalkyl” refers to the group —R-Het where Het is a heterocycle group and R is a alkyl group as defined above. Heteroalkyl groups can optionally be unsubstituted or substituted as defined above with e.g., halogen, amino, hydroxy, cyano, nitro, mercapto, alkoxy, alkylamino, acylamino, carbamoylamino, acyloxy, dialkylamino, alkylthio, carboxyl, amido, sulfo, sulfamido, and the like.

“Heteroarylalkyl” refers to the group —R-HetAr where HetAr is an heteroaryl group and R is alkyl or substituted alkyl as defined above. Heteroarylalkyl groups can optionally be unsubstituted or substituted as defined above with, e.g., halogen, alkyl, substituted alkyl, alkoxy, alkylthio, nitro, mercapto, sulfo and the like.

“Cycloalkyl” refers to a divalent cyclic or polycyclic alkyl group containing preferably 3 to 15 carbon atoms.

“Substituted cycloalkyl” refers to a cycloalkyl group comprising one or more substituents as defined above with, e.g., halogen, amino, hydroxy, cyano, nitro, mercapto, alkoxy, alkylamino, acylamino, carbamoylamino, acyloxy, dialkylamino, alkylthio, carboxyl, amido, sulfo, sulfamido, and the like.

“Cycloheteroalkyl” refers to a cycloalkyl group as defined above wherein one or more, preferably 1 to 3, of the ring methylene group is replaced with a heteroatom (e.g., NH, O, S)

“Substituted cycloheteroalkyl” refers to a cycloheteroalkyl group as herein defined which contains one or more substituents as defined above, such as halogen, amino, hydroxy, cyano, nitro, mercapto, alkoxy, alkylamino, acylamino, carbamoylamino, acyloxy, dialkylamino, alkylthio, carboxyl, amido, sulfo, sulfamido and the like.

“Cycloalkyl alkyl” denotes the group —R-cycloalkyl where cycloalkyl is a cycloalkyl group as defined above and R is an alkyl or substituted alkyl as defined above. Cycloalkyl groups can optionally be unsubstituted or substituted as defined above with e.g., halogen, amino, hydroxy, cyano, nitro, mercapto, alkoxy, alkylamino, acylamino, carbamoylamino, acyloxy, dialkylamino, alkylthio, carboxyl, amido, sulfo, sulfamido and the like.

“Cycloheteroalkyl alkyl” denotes he group —R-cycloheteroalkyl where R is a alkyl or substituted alkyl as defined above and cycloheteroalkyl as defined above. Cycloheteroalkyl groups can optionally be unsubstituted or substituted as defined above with e.g. halogen, amino, hydroxy, cyano, nitro, . mercapto, alkoxy, alkylamino, acylamino, carbamoylamino, acyloxy, dialkylamino, alkylthio, carboxyl, amido, sulfo, sulfamido, and the like.

In a preferred embodiment the invention relates to 3-,7-disubstituted pyrazolo[4,3-d]pyrimidines, which inhibit the cyclin-dependent and MAP kinases have formula I 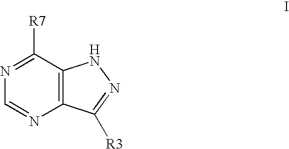
and the pharmaceutically acceptable acid salts thereof, wherein
R3 is
selected from an alkyl, cycloalkyl, cycloalkyl alkyl, cycloheteroalkyl, cycloheteroalkylalkyl, cycloalkyl alkyl, aryl, arylalkyl, heteroaryl, heteroarylalkyl, wherein each of the groups may be optionally be substituted by a halogen;
R7 is selected from the group consisting of halogen, hydroxyl, hydroxylamino, amino, hydrazino, carboxyl, cyano, nitro, amido, sulfo, sulfamido, carbamino, NHCONH2, NHC(═NH)NH2, alkyl, cycloalkyl, cycloalkylalkyl, aryl, arylalkyl, heteroalkyl, heteroaryl, heteroarylalkyl, cycloheteroalkyl, cycloheteroalkylalkyl, which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
R7′-X, wherein X is —NH—, —O—, —S—;
R7′-X, wherein X is preferably —N(alkyl)- selected at each occurrence from the group methyl, ethyl, propyl, isopropyl, ethinyl, allyl, propargyl, and isopent-2-en-1-yl;
   R7′ is
C₁-C₈ branched or unbranched alkyl, alkenyl or alkinyl preferentially selected from the group methyl, ethyl, isopropyl, butyl, isobutyl, allyl, propargyl, isopent-2-en-1-yl, which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
acyl, —C(O)R, wherein Rₐ is C₁-C₆ branched or unbranched alkyl, alkenyl or alkinyl preferentially selected from the group methyl, ethyl, isopropyl, butyl, isobutyl, allyl, propargyl, isopent-2-en-1-yl, and 2-methylallyl, which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
amido, —C(O)NR_{b}R_{c}, wherein R_{b} and R_{c} is independently H, C₁-C₆ branched or unbranched alkyl, alkenyl or alkinyl preferentially selected from the group methyl, ethyl, isopropyl, butyl, isobutyl, allyl, propargyl, which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
sulfo, —SO₃R_{d}, wherein R_{d} is H, C₁-C₆ branched or unbranched alkyl, alkenyl or alkinyl preferentially selected from the group methyl, ethyl, isopropyl, butyl, isobutyl, allyl, propargyl, isopent-2-en-1-yl, and 2-methylallyl which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
cycloalkyl is C₃-C₁₅ cycloalkyl is preferentially selected from the group cyclopropyl, cyclobutyl, cyclopentyl, cyclohexyl, cycloheptyl or adamantyl;
substituted cycloalkyl is C₃-C₁₅ cycloalkyl is preferentially selected from the group cyclopropyl, cyclobutyl, cyclopentyl, cyclohexyl, cycloheptyl or adamantyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
cycloalkyl alkyl is R_{f} (cycloalkyl), wherein R_{f} is
   C₁-C₆ alkyl, alkenyl or alkinyl preferentially selected from the group methyl, ethyl, isopropyl, butyl, allyl, propargyl, isopent-2-en-1-yl and 2-methylallyl, which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group,
   cykloalkyl is C₃-C₁₅ cycloalkyl is preferentially selected from the group cyclopropyl, cyclobutyl, cyclopentyl, cyclohexyl, cycloheptyl or adamantyl;
   substituted cycloalkyl is C₃-C₁₅ cycloalkyl is preferentially selected from the group cyclopropyl, cyclobutyl, cyclopentyl, cyclohexyl, cycloheptyl or adamantyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
   aryl is preferentially selected from the group phenyl, biphenyl, naphthyl, tetrahydronaphtyl, fluorenyl, indenyl or fenanthrenyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
   heterocycle is preferentially selected from the group thienyl, furyl, pyranyl, pyrrolyl, imidazolyl, pyrazolyl, pyridyl, pyrazinyl, pyrimidinyl, pyridazinyl, isothiazolyl, isoxazyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
heteroalkyl is —R_{g}-Het, wherein
   R_{g} is C₁-C₆ alkyl, alkenyl or alkinyl preferentially selected from the group methylen, 1,2-ethyliden, 1,3-propiliden, 1,4-butyliden, pentamethylen, hexamethylen, ethylendiyl, allyl-1,3-diyl, methylethan-1,1-diyl, methylethan-1,2-diyl, butan-1,3-diyl, which is substituted independently at each occurrence with 0-5 substituents selected from the group halogen, hydroxy, alkoxy, cyano and
   Het is preferentially selected from the group thienyl, furyl, pyranyl, pyrrolyl, imidazolyl, pyrazolyl, pyridyl, pyrazinyl, pyrimidinyl, pyridazinyl, isothiazolyl, isoxazyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
heteroaryl is —Rₕ-HetAr, wherein
   Rₕ is C₁-C₆ alkyl, alkenyl or alkinyl preferentially selected from the group methylen, 1,2-ethyliden, 1,3-propiliden, 1,4-butyliden, pentamethylen, hexamethylen, ethylendiyl, allyl-1,3-diyl, methylethan-1,1-diyl, methylethan-1,2-diyl, butan-1,3-diyl, which is substituted independently at each occurrence with 0-5 substituents selected from the group halogen, hydroxy, alkoxy, cyano;
   HetAr is preferentially selected from the group benzothienyl, naphthothienyl, benzofuranyl, chromenyl, indolyl, isoindolyl, indazolyl, qinolyl, isoqinolyl, ftalazinyl, qinaxalinyl, cinnolinyl, qinazolinyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
arylalkyl is —RᵢAr, wherein
   Rᵢ is C₁-C₆ alkyl, alkenyl or alkinyl preferentially selected from the group group methylen, 1,2-ethyliden, 1,3-propiliden, 1,4-butyliden, pentamethylen, hexamethylen, ethylendiyl, allyl-1,3-diyl methylethan-1,1-diyl, methylethan-1,2-diyl, butan-1,3-diyl, which is substituted independently at each occurrence with 0-5 substituents selected from the group halogen, hydroxy, alkoxy, cyano;
   Ar is preferentially selected from the group phenyl, biphenyl, naphthyl, tetrahydronaphtyl, fluorenyl, indenyl or fenanthrenyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
cycloheteroalkyl is preferentially selected from the group piperidinyl, piperazinyl, morfolinyl, pyrrolidinyl, imidazolidinyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
cycloheteroalkyl alkyl, —Rⱼ(cycloheteroalkyl), wherein
   Rⱼ is arylalkyl —RᵢAr, wherein
      Rᵢ is C₁-C₆ alkyl, alkenyl or alkinyl preferentially selected from the group group methylen, 1,2-ethyliden, 1,3-propiliden, 1,4-butyliden, pentamethylen, hexamethylen, ethylendiyl, allyl-1,3-diyl, methylethan-1,1-diyl, methylethan-1,2-diyl, butan-1,3-diyl, which is substituted independently at each occurrence with 0-5 substituents selected from the group halogen, hydroxy, alkoxy, cyano, and
      Ar is preferentially selected from the group phenyl, biphenyl, naphthyl, tetrahydronaphtyl, fluorenyl, indenyl or fenanthrenyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group, and
   cycloheteroalkyl is preferentially selected from the group piperidinyl, piperazinyl, morfolinyl, pyrrolidinyl, imidazolidinyl substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
heteroarylalkyl is —Rₖ-HetAr, wherein
   Rₖ is C₁-C₆ alkyl, alkenyl or alkinyl preferentially selected from the group group methylen, 1,2-ethyliden, 1,3-propiliden, 1,4-butyliden, pentamethylen, hexamethylen, ethylendiyl, allyl-1,3-diyl methylethan-1,1-diyl, methylethan-1,2-diyl, butan-1,3-diyl, which is substituted independently at each occurrence with 0-5 substituents selected from the group halogen, hydroxy, alkoxy, cyano, and
      HetAr is preferentially selected from the group benzothienyl, benzofuranyl, chromenyl, indolyl, isoindolyl, indazolyl, qinolinyl, phthalazinyl, qinoxalinyl, qinazolinyl, karbazolyl, akridinyl, indolinyl, and isoindolinyl, which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group.

The following derivatives are particularly preferred, namely: 7-(2-hydroxy-3-chlorobenzyl)amino-3-(methyl, ethyl, isopropyl, cyclopropyl, 3-pentyl, benzyl)pyrazolo[4,3-d]pyrimidine, 7-(2-hydroxy-4-chlorobenzyl)amino-3-(methyl, ethyl, isopropyl, cyclopropyl, 3-pentyl benzyl)pyrazolo[4,3-d]pyrimidine, 7-(2-hydroxy-5-chlorobenzyl)amino-3-(methyl, ethyl, isopropyl, cyclopropyl, 3-pentyl. benzyl)pyrazolo[4,3-d]pyrimidine, 7-(2-hydroxy-6-chlorobenzyl)amino-3-(methyl, ethyl, isopropyl, 3-pentyl benzyl)pyrazolo[4,3-d]pyrimidine, 7-(2-hydroxy-3-iodobenzyl)amino-3-(methyl, ethyl, isopropyl, 3-pentyl, benzyl)pyrazolo[4,3-d]pyrimidine, 7-(2-hydroxy-4-iodobenzyl)amino-3-(methyl, ethyl, isopropyl, 3-pentyl, benzyl)pyrazolo[4,3-d]pyrimidine, 7-(2-hydroxy-5-iodobenzyl)amino-3-(methyl, ethyl, isopropyl, 3-pentyl, benzyl)pyrazolo[4,3-d]pyrimidine, . 7-(2-hydroxy-6-iodobenzyl)amino-3-(methyl, ethyl, isopropyl, 3-pentyl, benzyl)pyrazolo[4,3-d]pyrimidine, 7-(2-hydroxy-3-bromobenzyl)amino-3-(methyl, ethyl, isopropyl, 3-pentyl, benzyl)pyrazolo[4,3-d]pyrimidine, 3-pentyl, 7-(2-hydroxy-4-bromobenzyl)amino-3-(methyl, ethyl, isopropyl, 3-pentyl, benzyl)pyrazolo[4,3-d]pyrimidine, 7-(2-hydroxy-5-bromobenzyl)amino-3-(methyl, ethyl, isopropyl, 3-pentyl, benzyl)pyrazolo[4,3-d]pyrimidine, 7-(2-hydroxy-6-bromobenzyl)amino-3-(methyl, ethyl, isopropyl, 3-pentyl, benzyl)pyrazolo[4,3-d]pyrimidine, 7-(2-hydroxy-3-fluorobenzyl)amino-3-(methyl, ethyl, isopropyl, 3-pentyl, benzyl)pyrazolo[4,3-d]pyrimidine, 7-(2-hydroxy-4-fluorobenzyl)amino-3-(methyl, ethyl, isopropyl, 3-pentyl, benzyl)pyrazolo[4,3-d]pyrimidine, 7-(2-hydroxy-5-fluorobenzyl)amino-3-(methyl, ethyl, isopropyl, 3-pentyl, benzyl)pyrazolo[4,3-d]pyrimidine, 6-(2-hydroxy-6-fluorobenzyl)amino-3-(methyl, ethyl, isopropyl, 3-pentyl, benzyl)pyrazolo[4,3-d]pyrimidine, 7-(2,3-dihydroxy-4-methoxybenzyl)amino-3-(methyl, ethyl, isopropyl, 3-pentyl, benzyl)pyrazolo[4,3-d]pyrimidine, 7-(2,5-dihydroxy-4-methoxybenzyl)amino-3-(methyl, ethyl, isopropyl, 3-pentyl, benzyl)pyrazolo[4,3-d]pyrimidine, 7-(2,6-dihydroxy-3-methoxybenzyl)amino-3-(methyl, ethyl, isopropyl, 3-pentyl, benzyl)pyrazolo[4,3-d]pyrimidine, 7-(2,3-dihydroxy-3-methoxybenzyl)amino-3-(methyl, ethyl, isopropyl, 3-pentyl, benzyl)pyrazolo[4,3-d]pyrimidine, 7-(2,5-dihydroxy-3-methoxybenzyl)amino-3-(methyl, ethyl, isopropyl, 3-pentyl, benzyl)pyrazolo[4,3-d]pyrimidine, 7-(2,6-dihydroxy-4-methoxybenzyl)amino-3-(methyl, ethyl, isopropyl, 3-pentyl, benzyl)pyrazolo[4,3-d]pyrimidine, 7-(2,3-dihydroxy-4-chlorobenzyl)amino-3-(methyl, ethyl, isopropyl, 3-pentyl, benzyl)pyrazolo[4,3-d]pyrimidine, 7-(2,3-dihydroxy-4-chlorobenzyl)amino-3-(methyl, ethyl, isopropyl, 3-pentyl, benzyl)pyrazolo[4,3-d]pyrimidine, 7-(2,5-dihydroxy-4-chlorobenzyl)amino-3-(methyl, ethyl, isopropyl, 3-pentyl, benzyl)pyrazolo[4,3-d]pyrimidine, 7-(2,6-dihydroxy-4-chlorobenzyl)amino-3-(methyl, ethyl, isopropyl, 3-pentyl, benzyl)pyrazolo[4,3-d]pyrimidine, 7-(2,6-dihydroxy-4-bromoxybenzyl)amino-3-(methyl, ethyl, isopropyl, 3-pentyl, benzyl)pyrazolo[4,3-d]pyrimidine, 7-(2,6-dihydroxy-4-iodobenzyl)amino-3-(methyl, ethyl, isopropyl, 3-pentyl, benzyl)pyrazolo[4,3-d]pyrimidine, 7-(2,6-dihydroxy-3-chlorobenzyl)amino-3-(methyl, ethyl, isopropyl, 3-pentyl, benzyl)pyrazolo[4,3-d]pyrimidine, 7-(2,6-dihydroxy-3-bromobenzyl)amino-3-(methyl, ethyl, isopropyl, 3-pentyl, benzyl)pyrazolo[4,3-d]pyrimidine, 7-(2,6-dihydroxy-3-iodobenzyl)amino-3-(methyl, ethyl, isopropyl, 3-pentyl, benzyl)pyrazolo[4,3-d]pyrimidine, 7-(2,6-dihydroxy-3-fluorobenzyl)amino-3-(methyl, ethyl, isopropyl, 3-pentyl, benzyl)pyrazolo[4,3-d]pyrimidine, 7-(2,6-dihydroxy-3,5-dichlorobenzyl)amino-3-(methyl, ethyl, isopropyl, 3-pentyl, benzyl)pyrazolo[4,3-d]pyrimidine, 7-(2,6-dihydroxy-3,5-dibromobenzyl)amino-3-(methyl, ethyl, isopropyl, 3-pentyl, benzyl)pyrazolo[4,3-d]pyrimidine, 7-(2,6-dihydroxy-3,5-diiodobenzyl)amino-3-(methyl, ethyl, isopropyl, 3-pentyl, benzyl)pyrazolo[4,3-d]pyrimidine, 7-(2,6-dihydroxy-3,5-difluorobenzyl)amino-3-(methyl, ethyl, isopropyl, 3-pentyl, benzyl)pyrazolo[4,3-d]pyrimidine, 7-(2-fluorobenzyl)amino-3-(methyl, ethyl, isopropyl, 3-pentyl, benzyl)pyrazolo[4,3-d]pyrimidine, 7-(3-fluorobenzyl)amino-3-(methyl, ethyl, isopropyl, 3-pentyl, benzyl)pyrazolo[4,3-d]pyrimidine, 7-(4-fluorobenzyl)amino-3-(methyl, ethyl, isopropyl, 3-pentyl, benzyl)pyrazolo[4,3-d]pyrimidine, 7-(2-bromobenzyl)amino-3-(methyl, ethyl, isopropyl, 3-pentyl, benzyl)pyrazolo[4,3-d]pyrimidine, 7-(3-bromobenzyl)amino-3-(methyl, ethyl, isopropyl, 3-pentyl, benzyl)pyrazolo[4,3-d]pyrimidine, 7-(4-bromobenzyl)amino-3-(methyl, ethyl, isopropyl, 3-pentyl, benzyl)pyrazolo[4,3-d]pyrimidine, 7-(2-iodobenzyl)amino-3-(methyl, ethyl, isopropyl, 3-pentyl, benzyl)pyrazolo[4,3-d]pyrimidine, 7-(3-iodobenzyl)amino-3-(methyl, ethyl, isopropyl, 3-pentyl, benzyl)pyrazolo[4,3-d]pyrimidine, 7-(4-iodobenzyl)amino-3-(methyl, ethyl, isopropyl, 3-pentyl, benzyl)pyrazolo[4,3-d]pyrimidine, 7-(2-chlorobenzyl)amino-3-(methyl, ethyl, isopropyl, 3-pentyl, benzyl)pyrazolo[4,3-d]pyrimidine, 7-(2-chlorobenzyl)amino-3-(methyl, ethyl, isopropyl, 3-pentyl, benzyl)pyrazolo[4,3-d]pyrimidine, 7-(3-chlorobenzylamino)-3-(methyl, ethyl, isopropyl, 3-pentyl, benzyl)pyrazolo[4,3-d]pyrimidine 7-(4-chlorobenzyl)amino-3-(methyl, ethyl, isopropyl, 3-pentyl, benzyl)pyrazolo[4,3-d]pyrimidine, 7-(2-acetylbenzyl)amino-3-(methyl, ethyl, isopropyl, 3-pentyl, benzyl)pyrazolo[4,3-d]pyrimidine, 7-(3-acetylbenzyl)amino-3-(methyl, ethyl, isopropyl, 3-pentyl, benzyl)pyrazolo[4,3-d]pyrimidine, 7-(4-acetylbenzyl)amino-3-(methyl, ethyl, isopropyl, 3-pentyl, benzyl)pyrazolo[4,3-d]pyrimidine, 7-(3-karboxybenzyl)amino-3-(methyl, ethyl, isopropyl, 3-pentyl, benzyl)pyrazolo[4,3-d]pyrimidine, 7-(4-karboxybenzyl)amino-3-(methyl, ethyl, isopropyl, 3-pentyl, benzyl)pyrazolo[4,3-d]pyrimidine, 7-(2-acetoxybenzyl)amino-3-(methyl, ethyl, isopropyl, 3-pentyl, benzyl)pyrazolo[4,3-d]pyrimidine, 7-(3-acetoxybenzyl)amino-3-(methyl, ethyl, isopropyl, 3-pentyl, benzyl)pyrazolo[4,3-d]pyrimidine, 7-(4-acetoxybenzyl)amino-3-(methyl, ethyl, isopropyl, 3-pentyl, benzyl)pyrazolo[4,3-d]pyrimidine, 7-(2-nitrobenzyl)amino-3-(methyl, ethyl, isopropyl, 3-pentyl, benzyl)pyrazolo[4,3-d]pyrimidine, 7-(3-nitrobenzyl)amino-3-(methyl, ethyl, isopropyl, 3-pentyl, benzyl)pyrazolo[4,3-d]pyrimidine, 7-(4-nitrobenzyl)amino-3-(methyl, ethyl, isopropyl, 3-pentyl, benzyl)pyrazolo[4,3-d]pyrimidine, 7-(2-cyanobenzyl)amino-3-(methyl, ethyl, isopropyl, 3-pentyl, benzyl)pyrazolo[4,3-d]pyrimidine, 7-(3-cyanobenzyl)amino-3-(methyl, ethyl, isopropyl, 3-pentyl, benzyl)pyrazolo[4,3-d]pyrimidine, 7-(4-cyanobenzyl)amino-3-(methyl, ethyl, isopropyl, 3-pentyl, benzyl)pyrazolo[4,3-d]pyrimidine, 7-(5-nitro-2-methylbenzyl)amino-3-(methyl, ethyl, isopropyl, benzyl)pyrazolo[4,3-d]pyrimidine, 7-(2-methylbenzyl)amino-3-(methyl, ethyl, isopropyl, benzyl)pyrazolo[4,3-d]pyrimidine, 7-(3-methylbenzyl)amino-3-(methyl, ethyl, isopropyl, benzyl)pyrazolo[4,3-d]pyrimidine, 7-(4-methylbenzyl)amino-3-(methyl, ethyl, isopropyl, 3-pentyl, benzyl)pyrazolo[4,3-d]pyrimidine, 7-(4-methylaminobenzyl)amino-3-(methyl, ethyl, isopropyl, 3-pentyl, benzyl)pyrazolo[4,3-d]pyrimidine, 7-(2-methoxybenzyl)amino-3-(methyl, ethyl, isopropyl, 3-pentyl, benzyl)pyrazolo[4,3-d]pyrimidine, 7-(3-methoxybenzyl)amino-3-(methyl, ethyl, isopropyl, 3-pentyl, benzyl)pyrazolo[4,3-d]pyrimidine, 7-(4-methoxybenzyl)amino-3-(methyl, ethyl, isopropyl, 3-pentyl, benzyl)pyrazolo[4,3-d]pyrimidine, 7-(2-hydroxybenzyl)amino-3-(methyl, ethyl, isopropyl, 3-pentyl, benzyl)pyrazolo[4,3-d]pyrimidine, 7-(3-hydroxybenzyl)amino-3-(methyl, ethyl, isopropyl, 3-pentyl, benzyl)pyrazolo[4,3-d]pyrimidine, 7-(4-hydroxybenzyl)amino-3-(methyl, ethyl, isopropyl, 3-pentyl, benzyl)pyrazolo[4,3-d]pyrimidine, 7-(4-hexylbenzyl)amino-3-(methyl, ethyl, isopropyl, 3-pentyl, benzyl)pyrazolo[4,3-d]pyrimidine, 7-(4-hexyloxybenzyl)amino-3-(methyl, ethyl, isopropyl, 3-pentyl, benzyl)pyrazolo[4,3-d]pyrimidine, 7-(2-ethoxybenzyl)amino)-3-(methyl, ethyl, isopropyl, 3-pentyl, benzyl)pyrazolo[4,3-d]pyrimidine, 7-(3-ethoxybenzyl)amino-3-(methyl, ethyl, isopropyl, 3-pentyl, benzyl)pyrazolo[4,3-d]pyrimidine, 7-(4-ethoxybenzyl)amino-3-(methyl, ethyl, isopropyl, 3-pentyl, benzyl)pyrazolo[4,3-d]pyrimidine, 7-(4-ethylbenzyl)amino-3-(methyl, ethyl, isopropyl, 3-pentyl, benzyl)pyrazolo[4,3-d]pyrimidine, 7-(4-penthylbenzyl)amino-3-(methyl, ethyl, isopropyl, 3-pentyl, benzyl)pyrazolo[4,3-d]pyrimidine, 7-(4-penthyloxybenzyl)amino-3-(methyl, ethyl, isopropyl, 3-pentyl, benzyl)pyrazolo[4,3-d]pyrimidine, 7-(4-fenoxybenzyl)amino-3-(methyl, ethyl, isopropyl, 3-pentyl, benzyl)pyrazolo[4,3-d]pyrimidine, 7-(4-fenylbenzyl)amino-3-(methyl, ethyl, isopropyl, 3-pentyl, benzyl)pyrazolo[4,3-d]pyrimidine, 7-(4-propylbenzyl)amino-3-(methyl, ethyl, isopropyl, 3-pentyl, benzyl)pyrazolo[4,3-d]pyrimidine, 7-(4-propyloxybenzyl)aminopurin, 7-(4-oktylbenzylamino)-3-(methyl, ethyl, isopropyl, 3-pentyl, benzyl)pyrazolo[4,3-d]pyrimidine, 7-(4-octyloxybenzyl)amino-3-(methyl, ethyl, isopropyl, 3-pentyl, benzyl)pyrazolo[4,3-d]pyrimidine, 7-(4-ethyloxybenzyl)amino-3-(methyl, ethyl, isopropyl, 3-pentyl, benzyl)pyrazolo[4,3-d]pyrimidine, 7-(3,4-diacetoxybenzyl)amino-3-(methyl, ethyl, isopropyl, 3-pentyl, benzyl)pyrazolo[4,3-d]pyrimidine, 7-(3,5-diacetoxybenzyl)amino-3-(methyl, ethyl, isopropyl, 3-pentyl, benzyl)pyrazolo[4,3-d]pyrimidine, 67-(2,5-diaminobenzyl)amino-3-(methyl, ethyl, isopropyl, 3-pentyl, benzyl)pyrazolo[4,3-d]pyrimidine, 7-(3,5-dibromobenzyl)amino-3-(methyl, ethyl, isopropyl, 3-pentyl, benzyl)pyrazolo[4,3-d]pyrimidine, 7-(3,5-dibromo-4-methoxybenzyl)amino-3-(methyl, ethyl, isopropyl, 3-pentyl, benzyl)pyrazolo[4,3-d]pyrimidine, 7-(2,3-dichlorobenzyl)amino-3-(methyl, ethyl, isopropyl, 3-pentyl, benzyl)pyrazolo[4,3-d]pyrimidine, 7-(2,4-dichlorobenzyl)amino-3-(methyl, ethyl, isopropyl, 3-pentyl, benzyl)pyrazolo[4,3-d]pyrimidine, 7-(2,5-dichlorobenzyl)amino-3-(methyl, ethyl, isopropyl, 3-pentyl, benzyl)pyrazolo[4,3-d]pyrimidine, 7-(2,6-dichlorobenzyl)amino-3-(methyl, ethyl, isopropyl, 3-pentyl, benzyl)pyrazolo[4,3-d]pyrimidine, 7-(3,4-dichlorobenzyl)amino 3-(methyl, ethyl, isopropyl, 3-pentyl, benzyl)pyrazolo[4,3-d]pyrimidine, 7-(3,5-dichlorobenzyl)amino-3-(methyl, ethyl, isopropyl, 3-pentyl, benzyl)pyrazolo[4,3-d]pyrimidine, 7-(2,3,4,5-tetrafluorobenzyl)amino-3-(methyl, ethyl, isopropyl, 3-pentyl, benzyl)pyrazolo[4,3-d]pyrimidine, 7-(2-chloro-3,6-difluorobenzyl)amino-3-(methyl, ethyl, isopropyl, 3-pentyl, benzyl)pyrazolo[4,3-d]pyrimidine, 7-(5-chloro-2-fluorobenzyl)amino-3-(methyl, ethyl, isopropyl, 3-pentyl, benzyl)pyrazolo[4,3-d]pyrimidine, 7-(2,3,4-trifluorobenzyl)amino-3-(methyl, ethyl, isopropyl, 3-pentyl, benzyl)pyrazolo[4,3-d]pyrimidine, 7-(2,3,5-trifluorobenzyl)amino-3-(methyl, ethyl, isopropyl, 3-pentyl, benzyl)pyrazolo[4,3-d]pyrimidine, 7-(2,4,5-trifluorobenzyl)amino-3-(methyl, ethyl, isopropyl, 3-pentyl, benzyl)pyrazolo[4,3-d]pyrimidine, 7-(3,4,5-trifluorobenzyl)amino-3-(methyl, ethyl, isopropyl, 3-pentyl, benzyl)pyrazolo[4,3-d]pyrimidine, 7-(2,3,6-trifluorobenzyl)amino-3-(methyl, ethyl, isopropyl, 3-pentyl, benzyl)pyrazolo[4,3-d]pyrimidine, 7-(3-chloro-2,6-difluorobenzyl)amino-3-(methyl, ethyl, isopropyl, 3-pentyl, benzyl)pyrazolo[4,3-d]pyrimidine, 7-(2-chloro-6-fluorobenzyl)amino-3-(methyl, ethyl, isopropyl, 3-pentyl, benzyl)pyrazolo[4,3-d]pyrimidine, 7-(2,6difluorobenzyl)amino-3-(methyl, ethyl, isopropyl, 3-pentyl, benzyl)pyrazolo[4,3-d]pyrimidine, 7-(2,4-difluorobenzyl)amino-3-(methyl, ethyl, isopropyl, 3-pentyl, benzyl)pyrazolo[4,3-d]pyrimidine, 7-(3,4-difluorobenzyl)amino-3-(methyl, ethyl, isopropyl, 3-pentyl, benzyl)pyrazolo[4,3-d]pyrimidine, 7-(2,5-difluorobenzyl)amino-3-(methyl, ethyl, isopropyl, 3-pentyl, benzyl)pyrazolo[4,3-d]pyrimidine, 7-(3,5-difluorobenzyl)amino-3-(methyl, ethyl, isopropyl, 3-pentyl, benzyl)pyrazolo[4,3-d]pyrimidine, 7-[5-fluoro-2-(trifluoromethyl)benzyl]amino-3-(methyl, ethyl, isopropyl, 3-pentyl, benzyl)pyrazolo[4,3-d]pyrimidine, 7-[4-fluoro-2-(trifluoromethyl)benzyl]amino-3-(methyl, ethyl, isopropyl, 3-pentyl, benzyl)pyrazolo[4,3-d]pyrimidine, 7-(2-chloro-5-(trifluoromethyl)benzyl)amino-3-(methyl, ethyl, isopropyl, 3-pentyl, benzyl)pyrazolo[4,3-d]pyrimidine, 7-(2-(difluoromethoxy)benzyl)amino-3-(methyl, ethyl, isopropyl, 3-pentyl, benzyl)pyrazolo[4,3-d]pyrimidine, 7-(3-(difluoromethoxy)benzyl)amino-3-(methyl, ethyl, isopropyl, 3-pentyl, benzyl)pyrazolo[4,3-d]pyrimidine, 7-(4-(difluoromethoxy)benzyl)amino-3-(methyl, ethyl, isopropyl, 3-pentyl, benzyl)pyrazolo[4,3-d]pyrimidine, 7-[2-fluoro-5-(trifluoromethyl)benzyl]amino-3-(methyl, ethyl, isopropyl, 3-pentyl, benzyl)pyrazolo[4,3-d]pyrimidine, 7-[3-fluoro-4-(trifluoromethyl)benzyl]amino-3-(methyl, ethyl, isopropyl, 3-pentyl, benzyl)pyrazolo[4,3-d]pyrimidine, 7-[2-fluoro-4-(trifluoromethyl)benzyl]amino-3-(methyl, ethyl, isopropyl, 3-pentyl, benzyl)pyrazolo[4,3-d]pyrimidine, 7-(2-(trifluoromethylthio)benzylamino)purin, 7-[2-fluoro-3-trifluoromethyl)benzy]lamino-3-(methyl, ethyl, isopropyl, 3-pentyl, benzyl)pyrazolo[4,3-d]pyrimidine, 7-(2-chloro-6-fluoro-3-methylbenzylamino)-3-(methyl, ethyl, isopropyl, 3-pentyl, benzyl)pyrazolo[4,3-d]pyrimidine, 7-(6-chloro-2-fluoro-3-methylbenzylamino)-3-(methyl, ethyl, isopropyl, 3-pentyl, benzyl)pyrazolo[4,3-d]pyrimidine, 7-[3-chloro-2-fluoro-5-(trifluoromethyl)benzyl]amino-3-(methyl, ethyl, isopropyl, 3-pentyl, benzyl)pyrazolo[4,3-d]pyrimidine, 7-[3-chloro-2-fluoro-6-(trifluoromethyl)benzyl]amino-3-(methyl, ethyl, isopropyl, 3-pentyl, benzyl)pyrazolo[4,3-d]pyrimidine, 7-(2,3-difluoro-4methylbenzyl)amino-3-(methyl, ethyl, isopropyl, 3-pentyl, benzyl)pyrazolo[4,3-d]pyrimidine, 7-(2,6-difluoro-3-methylbenzyl)amino-3-(methyl, ethyl, isopropyl, 3-pentyl, benzyl)pyrazolo[4,3-d]pyrimidine, 7-(2-fluoro-6-(trifluoromethyl)benzyl)amino-3-(methyl, ethyl, isopropyl, 3-pentyl, benzyl)pyrazolo[4,3-d]pyrimidine, 7-(3-chloro-2,6-difluorobenzyl)amino-3-(methyl, ethyl, isopropyl, 3-pentyl, benzyl)pyrazolo[4,3-d]pyrimidine, 7-[3-(trifluoromethylthio)benzy]lamino)-3-(methyl, ethyl, isopropyl, 3-pentyl, benzyl)pyrazolo[4,3-d]pyrimidine, 7-(3-fluoro-4-methylbenzyl)amino-3-(methyl, ethyl, isopropyl, 3-pentyl, benzyl)pyrazolo[4,3-d]pyrimidine, 7-[4-fluoro-3-methylbenzyl]amino-3-(methyl, ethyl, isopropyl, 3-pentyl, benzyl)pyrazolo[4,3-d]pyrimidine, 7-(5-fluoro-2-methylbenzylamino)-3-(methyl, ethyl, isopropyl, 3-pentyl, 3-pentyl, benzyl)pyrazolo[4,3-d]pyrimidine, 7-(2-chloro-3,6-difluorobenzyl)amino-3-(methyl, ethyl, isopropyl, 3-pentyl, benzyl)pyrazolo[4,3-d]pyrimidine, 7-[4-trifluoromethylthio)benzyl]amino-3-(methyl, ethyl, isopropyl, 3-pentyl, benzyl)pyrazolo[4,3-d]pyrimidine, 7-(3-fluoro-5-(trifluoromethyl)benzyl)amino-3-(methyl, ethyl, isopropyl, 3-pentyl, benzyl)pyrazolo[4,3-d]pyrimidine, 7-(2-chloro-4-fluorobenzyl)amino-3-(methyl, ethyl, isopropyl, 3-pentyl, benzyl)pyrazolo[4,3-d]pyrimidine, 7-(2-(trifluoromethoxy)benzyl)amino-3-(methyl, ethyl, isopropyl, 3-pentyl, benzyl)pyrazolo[4,3-d]pyrimidine, 7-(3-(trifluoromethyl)benzyl)amino-3-(methyl, ethyl, isopropyl, 3-pentyl, benzyl)pyrazolo[4,3-d]pyrimidine, 7-[2-(trifluoromethyl)benzyl]amino-3-(methyl, ethyl, isopropyl, 3-pentyl, benzyl)pyrazolo[4,3-d]pyrimidine, 7-(4-trifluoromethyl)benzyl)amino-3-(methyl, ethyl, isopropyl, 3-pentyl, benzyl)pyrazolo[4,3-d]pyrimidine, 7-[4-chloro-3-(trifluoromethyl)benzyl]amino)-3-(methyl, ethyl, isopropyl, 3-pentyl, benzyl)pyrazolo[4,3-d]pyrimidine, 7-(4-fluoro-3-(trifluoromethyl)benzyl)amino-3-(methyl, ethyl, isopropyl, 3-pentyl, benzyl)pyrazolo[4,3-d]pyrimidine, 7-[3,5-di(trifluoromethyl)benzyl]amino-3-(methyl, ethyl, isopropyl, 3-pentyl, benzyl)pyrazolo[4,3-d]pyrimidine, 7-[3-(trifluoromethoxy)benzyl]amino-3-(methyl, ethyl, isopropyl, 3-pentyl, benzyl)pyrazolo[4,3-d]pyrimidine, 7-[4-(trifluoromethoxy)benzyl]amino-3-(methyl, ethyl, isopropyl, 3-pentyl, benzyl)pyrazolo[4,3-d]pyrimidine, 7-(4-(trifluoromethyl)benzyl)amino-3-(methyl, ethyl, isopropyl, 3-pentyl, benzyl)pyrazolo[4,3-d]pyrimidine, 7-[4-(diethylamino)benzyl]amino-3-(methyl, ethyl, isopropyl, 3-pentyl, benzyl)pyrazolo[4,3-d]pyrimidine, 7-(3,4-dihydroxybenzyl]amino-3-(methyl, ethyl, isopropyl, 3-pentyl, benzyl)pyrazolo[4,3-d]pyrimidine, 7-(3,5-dihydroxybenzyl]amino-3-(methyl, ethyl, isopropyl, 3-pentyl, benzyl)pyrazolo[4,3-d]pyrimidine, 7-(3,4-dihydroxybenzyl]amino-3-(methyl, ethyl, isopropyl, 3-pentyl, benzyl)pyrazolo[4,3-d]pyrimidine, 7-(2,3-ethylenedioxybenzyl)amino-3-(methyl, ethyl, isopropyl, 3-pentyl, benzyl)pyrazolo[4,3-d]pyrimidine, 7-(2,4-dihydroxybenzyl)amino-3-(methyl, ethyl, isopropyl, 3-pentyl, benzyl)pyrazolo[4,3-d]pyrimidine, 7-(2,5-dihydroxybenzyl)amino-3-(methyl, ethyl, isopropyl, 3-pentyl, isopropyl, benzyl)pyrazolo[4,3-d]pyrimidine, 7-(2,6-dihydroxybenzyl)amino-3-(methyl, ethyl, isopropyl, 3-pentyl, benzyl)pyrazolo[4,3-d]pyrimidine, 7-(3,4-dimethoxybenzyl)amino-3-(methyl, ethyl, isopropyl, 3-pentyl, benzyl)pyrazolo[4,3-d]pyrimidine, 7-(3,4-dimethoxybenzyl)amino-3-(methyl, ethyl, isopropyl, 3-pentyl, benzyl)pyrazolo[4,3-d]pyrimidine, 7-(3,5-dimethoxybenzyl)amino-3-(methyl, ethyl, isopropyl, 3-pentyl, benzyl)pyrazolo[4,3-d]pyrimidine, 7-(2,3-dimethoxybenzyl)amino-3-(methyl, ethyl, isopropyl, 3-pentyl, benzyl)pyrazolo[4,3-d]pyrimidine, 7-(2,4-dimethoxybenzyl)amino-3-(methyl, ethyl, isopropyl, 3-pentyl, benzyl)pyrazolo[4,3-d]pyrimidine, 7-(2,5-dimethoxybenzyl)amino-3-(methyl, ethyl, isopropyl, 3-pentyl, benzyl)pyrazolo[4,3-d]pyrimidine, 7-(2,6-dimethoxybenzyl)amino-3-(methyl, ethyl, isopropyl, 3-pentyl, benzyl)pyrazolo[4,3-d]pyrimidine, 7-(3-hydroxy-4-methoxybenzyl)amino-3-(methyl, ethyl, isopropyl, 3-pentyl, benzyl)pyrazolo[4,3-d]pyrimidine, 7-(2-hydroxy-3-methoxybenzyl)amino-3-(methyl, ethyl, isopropyl, 3-pentyl, benzyl)pyrazolo[4,3-d]pyrimidine, 7-(4-hydroxy-3-methoxybenzyl)amino-3-(methyl, ethyl, isopropyl, 3-pentyl, benzyl)pyrazolo[4,3-d]pyrimidine, 7-(2-hydroxy-4-methoxybenzyl)amino-3-(methyl, ethyl, isopropyl, 3-pentyl, benzyl)pyrazolo[4,3-d]pyrimidine, 7-(4-hydroxy-2-methoxybenzyl)amino-3-(methyl, ethyl, isopropyl, 3-pentyl, benzyl)pyrazolo[4,3-d]pyrimidine, 7-(2-hydroxy-5-methoxybenzyl)amino-3-(methyl, ethyl, isopropyl, 3-pentyl, benzyl)pyrazolo[4,3-d]pyrimidine, 7-(3-hydroxy-4-methoxybenzyl)amino-3-(methyl, ethyl, isopropyl, 3-pentyl, benzyl)pyrazolo[4,3-d]pyrimidine, 7-(4-hydroxy-3-methoxybenzylamino)-3-(methyl, ethyl, isopropyl, 3-pentyl, benzyl)pyrazolo[4,3-d]pyrimidine, 7-(2-hydroxy-6-methoxybenzyl)amino-3-(methyl, ethyl, isopropyl, 3-pentyl, benzyl)pyrazolo[4,3-d]pyrimidine, 7-(3-hydroxy-5-methoxybenzyl)amino-3-(methyl, ethyl, isopropyl, 3-pentyl, benzyl)pyrazolo[4,3-d]pyrimidine, 7-(4,5-dimethoxy-2-nitrobenzyl)amino-3-(methyl, ethyl, isopropyl, 3-pentyl, benzyl)pyrazolo[4,3-d]pyrimidine, 7-(3,4-dimethylbenzyl)amino-3-(methyl, ethyl, isopropyl, 3-pentyl, benzyl)pyrazolo[4,3-d]pyrimidine, 7-(2,3-dimethylbenzyl)amino-3-(methyl, ethyl, isopropyl, 3-pentyl, benzyl)pyrazolo[4,3-d]pyrimidine, 7-(2,4-dimethylbenzyl)amino-3-(methyl, ethyl, isopropyl, 3-pentyl, benzyl)pyrazolo[4,3-d]pyrimidine, 7-(2,6-dimethylbenzyl)amino-3-(methyl, ethyl, isopropyl, 3-pentyl, benzyl)pyrazolo[4,3-d]pyrimidine, 7-(2,6-dimethyl-4-hydroxybenzyl)amino-3-(methyl, ethyl, isopropyl, 3-pentyl, benzyl)pyrazolo[4,3-d]pyrimidine, 7-(3,5-dimethyl-4-hydroxybenzyl)amino-3-(methyl, ethyl, isopropyl, 3-pentyl, benzyl)pyrazolo[4,3-d]pyrimidine, 7-(2-fluoro-4-hydroxybenzyl)amino-3-(methyl, ethyl, isopropyl, 3-pentyl, benzyl)pyrazolo[4,3-d]pyrimidine, 7-(3-fluoro-4-methylbenzyl)amino-3-(methyl, ethyl, isopropyl, 3-pentyl, benzyl)pyrazolo[4,3-d]pyrimidine, 7-(3,4-dinitrobenzyl)amino-3-(methyl, ethyl, isopropyl, 3-pentyl, benzyl)pyrazolo[4,3-d]pyrimidine, 7-(3,5-dinitrobenzyl)amino-3-(methyl, ethyl, isopropyl, 3-pentyl, benzyl)pyrazolo[4,3-d]pyrimidine, 7-(2-methyl-5-nitrobenzyl)amino-3-(methyl, ethyl, isopropyl, 3-pentyl, benzyl)pyrazolo[4,3-d]pyrimidine, 7-(3-methyl-4-nitrobenzyl)amino-3-(methyl, ethyl, isopropyl, 3-pentyl, benzyl)pyrazolo[4,3-d]pyrimidine, 7-(3,4-diiodo-4-hydroxybenzyl)amino-3-(methyl, ethyl, isopropyl, 3-pentyl, benzyl)pyrazolo[4,3-d]pyrimidine, 7-(2-chloro-3,4-dimethoxybenzyl)amino-3-(methyl, ethyl, isopropyl, 3-pentyl, benzyl)pyrazolo[4,3-d]pyrimidine, 7-(4-chloro-3,5-dinitrobenzyl)amino-3-(methyl, ethyl, isopropyl, 3-pentyl, benzyl)pyrazolo[4,3-d]pyrimidine, 7-(2-chloro-4-fluorobenzyl)amino-3-(methyl, ethyl, isopropyl, 3-pentyl, benzyl)pyrazolo[4,3-d]pyrimidine, 7-(3-chloro-4-fluorobenzyl)amino-3-(methyl, ethyl, isopropyl, 3-pentyl, benzyl)pyrazolo[4,3-d]pyrimidine, 7-(2-chloro-6-methylbenzyl)amino-3-(methyl, ethyl, isopropyl, benzyl)pyrazolo[4,3-d]pyrimidine, 7-(3-chloro-2-methylbenzyl)amino-3-(methyl, ethyl, isopropyl, 3-pentyl, benzyl)pyrazolo[4,3-d]pyrimidine, 7-(3-chloro-4-methylbenzyl)amino-3-(methyl, ethyl, isopropyl, 3-pentyl, benzyl)pyrazolo[4,3-d]pyrimidine, 7-(5-chloro-2-methoxybenzyl)amino-3-(methyl, ethyl, isopropyl, 3-pentyl, benzyl)pyrazolo[4,3-d]pyrimidine, 7-(2-chloro-4-fluorobenzyl)amino-3-(methyl, ethyl, isopropyl, 3-pentyl, benzyl)pyrazolo[4,3-d]pyrimidine, 7-(4-chloromethylbenzyl)amino-3-(methyl, ethyl, isopropyl, 3-pentyl, benzyl)pyrazolo[4,3-d]pyrimidine, 7-(2-chloro-5-nitrobenzyl)amino-3-(methyl, ethyl, isopropyl, 3-pentyl, benzyl)pyrazolo[4,3-d]pyrimidine, 7-(2-chloro-6-nitrobenzyl)amino-3-(methyl, ethyl, isopropyl, 3-pentyl, benzyl)pyrazolo[4,3-d]pyrimidine, 7-(4-chloro-3-nitrobenzyl)amino-3-(methyl, ethyl, isopropyl, 3-pentyl, benzyl)pyrazolo[4,3-d]pyrimidine, 7-(5-chloro-2-nitrobenzyl)amino-3-(methyl, ethyl, isopropyl, 3-pentyl, benzyl)pyrazolo[4,3-d]pyrimidine, 7-(3-bromo-4-hydroxybenzyl)amino-3-(methyl, ethyl, isopropyl, 3-pentyl, benzyl)pyrazolo[4,3-d]pyrimidine, 7-(3,5-dibromo-4-hydroxybenzyl)amino-3-(methyl, ethyl, isopropyl, 3-pentyl, benzyl)pyrazolo[4,3-d]pyrimidine, 7-(3-bromo-4-methoxybenzyl)amino-3-(methyl, ethyl, isopropyl, 3-pentyl, benzyl)pyrazolo[4,3-d]pyrimidine, 7-(4-butoxybenzyl)amino-3-(methyl, ethyl, isopropyl, 3-pentyl, benzyl)pyrazolo[4,3-d]pyrimidine, 7-(4-butoxybenzyl)amino-3-(methyl, ethyl, isopropyl, 3-pentyl, benzyl)pyrazolo[4,3-d]pyrimidine, 7-(4-/t-butyl/benzyl)amino-3-(methyl, ethyl, isopropyl, 3-pentyl, benzyl)pyrazolo[4,3-d]pyrimidine, 7-(4-t-butyl-2,6-dimethylbenzyl)amino-3-(methyl, ethyl, isopropyl, 3-pentyl, benzyl)pyrazolo[4,3-d]pyrimidine, 7-(2-aminobenzyl)amino-3-(methyl, ethyl, isopropyl, 3-pentyl, benzyl)pyrazolo[4,3-d]pyrimidine, 7-(3-aminobenzyl)amino-3-(methyl, ethyl, isopropyl, 3-pentyl, benzyl)pyrazolo[4,3-d]pyrimidine, 7-(4-aminobenzyl)amino-3-(methyl, ethyl, isopropyl, 3-pentyl, benzyl)pyrazolo[4,3-d]pyrimidine, 7-(2-amino-6-chlorobenzyl)amino-3-(methyl, ethyl, isopropyl, 3-pentyl, benzyl)pyrazolo[4,3-d]pyrimidine, 7-(3-amino-4-chlorobenzyl)amino-3-(methyl, ethyl, isopropyl, 3-pentyl, benzyl)pyrazolo[4,3-d]pyrimidine, 7-(2-amino-3-chlorobenzyl)amino-3-(methyl, ethyl, isopropyl, 3-pentyl, benzyl)pyrazolo[4,3-d]pyrimidine, 7-(2-amino-4-chlorobenzyl)amino-3-(methyl, ethyl, isopropyl, 3-pentyl, benzyl)pyrazolo[4,3-d]pyrimidine, 7-(2-amino-6-chlorobenzyl)amino-3-(methyl, ethyl, isopropyl, 3-pentyl, benzyl)pyrazolo[4,3-d]pyrimidine, 7-(2,6-diamino-3-chlorobenzyl)amino-3-(methyl, ethyl, isopropyl, 3-pentyl, benzyl)pyrazolo[4,3-d]pyrimidine, 7-(2,6-diamino-4-chlorobenzyl)amino-3-(methyl, ethyl, isopropyl, 3-pentyl, benzyl)pyrazolo[4,3-d]pyrimidine, 7-(4-amino-3-chlorobenzyl)amino-3-(methyl, ethyl, isopropyl, benzyl)pyrazolo[4,3-d]pyrimidine, 7-(4-amino-5-dichlorobenzyl)amino-3-(methyl, ethyl, isopropyl, 3-pentyl, benzyl)pyrazolo[4,3-d]pyrimidine, 7-(5-amino-2-methylbenzyl)amino-3-(methyl, ethyl, isopropyl, 3-pentyl, benzyl)pyrazolo[4,3-d]pyrimidine, 7-(2-amino-3-nitrobenzyl)amino-3-(methyl, ethyl, isopropyl, 3-pentyl, benzyl)pyrazolo[4,3-d]pyrimidine, 7-(4-amino-3-nitrobenzyl)amino-3-(methyl, ethyl, isopropyl, 3-pentyl, benzyl)pyrazolo[4,3-d]pyrimidine, 7-(4-benzyloxybenzyl)amino-3-(methyl, ethyl, isopropyl, 3-pentyl, benzyl)pyrazolo[4,3-d]pyrimidine, 7-(3-acetylbenzyl)amino-3-(methyl, ethyl, isopropyl, 3-pentyl, benzyl)pyrazolo[4,3-d]pyrimidine, 7-(2-acetylbenzyl)amino-3-(methyl, ethyl, isopropyl, 3-pentyl, benzyl)pyrazolo[4,3-d]pyrimidine, 7-(2,3,4-trimethoxybenzyl)amino-3-(methyl, ethyl, isopropyl, 3-pentyl, benzyl)pyrazolo[4,3-d]pyrimidine, 7-(2,4,5-trimethoxybenzyl)amino-3-(methyl, ethyl, isopropyl, 3-pentyl, benzyl)pyrazolo[4,3-d]pyrimidine, 7-(2,4,6-trimethoxybenzyl)amino-3-(methyl, ethyl, isopropyl, 3-pentyl, benzyl)pyrazolo[4,3-d]pyrimidine, 7-(3,4,5-trimethoxybenzyl)amino-3-(methyl, ethyl, isopropyl, 3-pentyl, benzyl)pyrazolo[4,3-d]pyrimidine, 7-(2,4,6-trimethoxybenzyl)amino-3-(methyl, ethyl, isopropyl, 3-pentyl, benzyl)pyrazolo[4,3-d]pyrimidine, 7-(2,3,4-trihydroxybenzyl)amino-3-(methyl, ethyl, isopropyl, 3-pentyl, benzyl)pyrazolo[4,3-d]pyrimidine, 7-(2,4,6-trihydroxybenzyl)lamino-3-(methyl, ethyl, isopropyl, 3-pentyl, benzyl)pyrazolo[4,3-d]pyrimidine, 7-(2,3,4-trihydroxybenzyl)amino-3-(methyl, ethyl, isopropyl, 3-pentyl, benzyl)pyrazolo[4,3-d]pyrimidine, 7-(3,4,5-trihydroxybenzyl)amino-3-(methyl, ethyl, isopropyl, 3-pentyl, benzyl)pyrazolo[4,3-d]pyrimidine, 7-(2,4,6-trihydroxybenzyl)amino-3-(methyl, ethyl, isopropyl, 3-pentyl, benzyl)pyrazolo[4,3-d]pyrimidine, 7-(2,4,5-trichlorobenzyl)amino-3-(methyl, ethyl, isopropyl, 3-pentyl, benzyl)pyrazolo[4,3-d]pyrimidine, 7-(2,4,5-trichlorobenzyl)amino-3-(methyl, ethyl, isopropyl, 3-pentyl, benzyl)pyrazolo[4,3-d]pyrimidine, 7-(2,4,6-trichlorobenzyl)amino-3-(methyl, ethyl, isopropyl, 3-pentyl, benzyl)pyrazolo[4,3-d]pyrimidine, 7-(2,3,4-trichlorobenzyl)amino-3-(methyl, ethyl, isopropyl, 3-pentyl, benzyl)pyrazolo[4,3-d]pyrimidine, 7-(2,3,5-trichlorobenzyl)amino-3-(methyl, ethyl, isopropyl, 3-pentyl, benzyl)pyrazolo[4,3-d]pyrimidine, 7-(2,3,6-trichlorobenzyl)amino-3-(methyl, ethyl, isopropyl, 3-pentyl, benzyl)pyrazolo[4,3-d]pyrimidine, 7-(2,5,6-trichlorobenzyl)amino-3-(methyl, ethyl, isopropyl, 3-pentyl, benzyl)pyrazolo[4,3-d]pyrimidine, 7-anilino-3-(methyl, ethyl, isopropyl, 3-pentyl, benzyl)pyrazolo[4,3-d]pyrimidine, 7-[2,4-bis(trifluoromethyl)anilino]-3-(methyl, ethyl, isopropyl, 3-pentyl, benzyl)pyrazolo[4,3-d]pyrimidine, 7-[2,5-bis(trifluoromethyl)anilino]-3-(methyl, ethyl, isopropyl, 3-pentyl, benzyl)pyrazolo[4,3-d]pyrimidine, 7-[2,4-bis(trifluoromethyl)anilino]-3-(methyl, ethyl, isopropyl, benzyl)pyrazolo[4,3-d]pyrimidine, 7-(3,5-bis(trifluoromethyl)anilino)-3-(methyl, ethyl, isopropyl, 3-pentyl, benzyl)pyrazolo[4,3-d]pyrimidine, 7-(2-bromoanilino)-3-(methyl, ethyl, isopropyl, 3-pentyl, benzyl)pyrazolo[4,3-d]pyrimidine, 7-(3-bromoanilino)-3-(methyl, ethyl, isopropyl, benzyl)pyrazolo[4,3-d]pyrimidine, 7-(4-bromoanilino)-3-(methyl, ethyl, isopropyl, 3-pentyl, benzyl)pyrazolo[4,3-d]pyrimidine, 7-(4-bromo-2-chloroanilino)-3-(methyl, ethyl, isopropyl, 3-pentyl, benzyl)pyrazolo[4,3-d]pyrimidine, 7-(4-bromo-3-chloroanilino)-3-(methyl, ethyl, isopropyl, 3-pentyl, benzyl)pyrazolo[4,3-d]pyrimidine, 7-[2-bromo-6-chloro-4-(trifluoromethyl)anilino]-3-(methyl, ethyl, isopropyl, 3-pentyl, benzyl)pyrazolo[4,3-d]pyrimidine, 7-(4-bromo-5,6-difluoroanilino)-3-(methyl, ethyl, isopropyl, 3-pentyl, benzyl)pyrazolo[4,3-d]pyrimidine, 7-(2-bromo-4,6-difluoroanilino)-3-(methyl, ethyl, isopropyl, 3-pentyl, benzyl)pyrazolo[4,3-d]pyrimidine, 7-(4-bromo-2,6-difluoroanilino)-3-(methyl, ethyl, isopropyl, 3-pentyl, benzyl)pyrazolo[4,3-d]pyrimidine, 7-(4-bromo-2-fluoroanilino)-3-(methyl, ethyl, isopropyl, 3-pentyl, benzyl)pyrazolo[4,3-d]pyrimidine, 7-(2-bromo-4-fluoroanilino)-3-(methyl, ethyl, isopropyl, 3-pentyl, benzyl)pyrazolo[4,3-d]pyrimidine, 7-(2-bromo-4-methylanilino)-3-(methyl, ethyl, isopropyl, 3-pentyl, benzyl)pyrazolo[4,3-d]pyrimidine, 7-(3-bromo-2-methylanilino)-3-(methyl, ethyl, isopropyl, 3-pentyl, benzyl)pyrazolo[4,3-d]pyrimidine, 6-(4-bromo-3-methylanilino))-3-(methyl, ethyl, isopropyl, 3-pentyl, benzyl)pyrazolo[4,3-d]pyrimidine, 7-[2-bromo-4-(trifluoromethoxy)anilino]-3-(methyl, ethyl, isopropyl, 3-pentyl, benzyl)pyrazolo[4,3-d]pyrimidine, 7-[3-bromo-4-(trifluoromethoxy)anilino]-3-(methyl, ethyl, isopropyl, 3-pentyl, benzyl)pyrazolo[4,3-d]pyrimidine, 7-[4-bromo-2-(trifluoromethoxy)anilino]-3-(methyl, ethyl, isopropyl, 3-pentyl, benzyl)pyrazolo[4,3-d]pyrimidine, 7-(2-bromo-4,5,6-trifluoroanilino)-3-(methyl, ethyl, isopropyl, 3-pentyl, benzyl)pyrazolo[4,3-d]pyrimidine, 7-(2,4-dibromoanilino)-3-(methyl, ethyl, isopropyl, 3-pentyl, benzyl)pyrazolo[4,3-d]pyrimidine, 7-(2,5-dibromoanilino)-3-(methyl, ethyl, isopropyl, 3-pentyl, benzyl)pyrazolo[4,3-d]pyrimidine, 7-(2,4-dibromo-3,6-dichloroanilino)-3-(methyl, ethyl, isopropyl, 3-pentyl, benzyl)pyrazolo[4,3-d]pyrimidine, 7-(2,6-dibromo-4-fluoroanilino)-3-(methyl, ethyl, isopropyl, 3-pentyl, benzyl)pyrazolo[4,3-d]pyrimidine, 7-[2,6-dibromo-4-(trifluoromethoxy)anilino]-3-(methyl, ethyl, isopropyl, 3-pentyl, benzyl)pyrazolo[4,3-d]pyrimidine, 7-[2,4-dibromo-6-(trifluoromethyl)anilino]-3-(methyl, ethyl, isopropyl, 3-pentyl, benzyl)pyrazolo[4,3-d]pyrimidine, 7-[2,6-dibromo-4-(trifluoromethyl)anilino]-3-(methyl, ethyl, isopropyl, 3-pentyl, benzyl)pyrazolo[4,3-d]pyrimidine, 7-(2,3-dichloroanilino)-3-(methyl, ethyl, isopropyl, 3-pentyl, benzyl)pyrazolo[4,3-d]pyrimidine, 7-(2,4-dichloroanilino)-3-(methyl, ethyl, isopropyl, 3-pentyl, benzyl)pyrazolo[4,3-d]pyrimidine, 7-(2,5-dichloroanilino)-3-(methyl, ethyl, isopropyl, 3-pentyl, benzyl)pyrazolo[4,3-d]pyrimidine, 7-(2,6-dichlorooanilino)-3-(methyl, ethyl, isopropyl, 3-pentyl, benzyl)pyrazolo[4,3-d]pyrimidine, 7-(3,4-dichloroanilino)-3-(methyl, ethyl, isopropyl, 3-pentyl, benzyl)pyrazolo[4,3-d]pyrimidine, 7-(3,5-dichloroanilino)-3-(methyl, ethyl, isopropyl, 3-pentyl, benzyl)pyrazolo[4,3-d]pyrimidine, 7-[2,6-dichloro-4-(trifluoromethoxy)anilino]-3-(methyl, ethyl, isopropyl, 3-pentyl, benzyl)pyrazolo[4,3-d]pyrimidine, 7-[2,4-dichloro-6-(trifluoromethyl)anilino]-3-(methyl, ethyl, isopropyl, 3-pentyl, benzyl)pyrazolo[4,3-d]pyrimidine, 7-[2,6-dichloro-4-(trifluoromethyl)anilino]-3-(methyl, ethyl, isopropyl, 3-pentyl, benzyl)pyrazolo[4,3-d]pyrimidine, 7-(2,3-difluoroanilino)-3-(methyl, ethyl, isopropyl, 3-pentyl, benzyl)pyrazolo[4,3-d]pyrimidine, 7-(2,4-difluoroanilino)-3-(methyl, ethyl, isopropyl, 3-pentyl, benzyl)pyrazolo[4,3-d]pyrimidine, 7-(2,5-difluoroanilino)-3-(methyl, ethyl, isopropyl, 3-pentyl, benzyl)pyrazolo[4,3-d]pyrimidine, 7-(2,6-difluoroanilino)-3-(methyl, ethyl, isopropyl, 3-pentyl, benzyl)pyrazolo[4,3-d]pyrimidine, 7-(3,4-difluoroanilino)-3-(methyl, ethyl, isopropyl, 3-pentyl, benzyl)pyrazolo[4,3-d]pyrimidine, 7-(3,5-difluoroanilino)-3-(methyl, ethyl, isopropyl, 3-pentyl, benzyl)pyrazolo[4,3-d]pyrimidine, 7-(2-difluoromethoxyanilino)-3-(methyl, ethyl, isopropyl, 3-pentyl, benzyl)pyrazolo[4,3-d]pyrimidine, 7-(2-difluoromethoxy-5-nitroanilino)-3-(methyl, ethyl, isopropyl, 3-pentyl, benzyl)pyrazolo[4,3-d]pyrimidine, 7-(2,3-difluoro-6-nitroanilino)-3-(methyl, ethyl, isopropyl, 3-pentyl, benzyl)pyrazolo[4,3-d]pyrimidine, 7-(2,4-difluoro-6-nitroanilino)-3-(methyl, ethyl, isopropyl, 3-pentyl, benzyl)pyrazolo[4,3-d]pyrimidine, 7-(2,4-dijodoanilino)-3-(methyl, ethyl, isopropyl, 3-pentyl, benzyl)pyrazolo[4,3-d]pyrimidine, 7-(2,3-dimethylanilino)-3-(methyl, ethyl, isopropyl, 3-pentyl, benzyl)pyrazolo[4,3-d]pyrimidine, 7-(2,4-dimethylanilino)-3-(methyl, ethyl, isopropyl, 3-pentyl, benzyl)pyrazolo[4,3-d]pyrimidine, 7-(2,3-dimethyl-6-nitroanilino)-3-(methyl, ethyl, isopropyl, 3-pentyl, benzyl)pyrazolo[4,3-d]pyrimidine, 7-(2,4-dimethoxyanilino)-3-(methyl, ethyl, isopropyl, 3-pentyl, benzyl)pyrazolo[4,3-d]pyrimidine, 7-(2,3-dimethoxyanilino)-3-(methyl, ethyl, isopropyl, 3-pentyl, benzyl)pyrazolo[4,3-d]pyrimidine, 7-(2,3-dinitro-6-methylanilino)-3-(methyl, ethyl, isopropyl, 3-pentyl, benzyl)pyrazolo[4,3-d]pyrimidine, 7-(4-hydroxy-2-methylanilino)-3-(methyl, ethyl, isopropyl, 3-pentyl, benzyl)pyrazolo[4,3-d]pyrimidine, 7-(2-chloroanilino)-3-(methyl, ethyl, isopropyl, 3-pentyl, benzyl)pyrazolo[4,3-d]pyrimidine, 7-(3-chloroanilino)-3-(methyl, ethyl, isopropyl, 3-pentyl, benzyl)pyrazolo[4,3-d]pyrimidine, 7-(4-chloroanilino)-3-(methyl, ethyl, isopropyl, 3-pentyl, benzyl)pyrazolo[4,3-d]pyrimidine, (7-chloro-2,6-dibromo-4-fluoroanilino)-3-(methyl, ethyl, isopropyl, 3-pentyl, benzyl)pyrazolo[4,3-d]pyrimidine, 7-(2-chloro-4-fluoroanilino)-3-(methyl, ethyl, isopropyl, 3-pentyl, benzyl)pyrazolo[4,3-d]pyrimidine, 7-(2-chloro-5-fluoroanilino)-3-(methyl, ethyl, isopropyl, 3-pentyl, benzyl)pyrazolo[4,3-d]pyrimidine, 7-(2-chloro-6-fluoroanilino)-3-(methyl, ethyl, isopropyl, 3-pentyl, benzyl)pyrazolo[4,3-d]pyrimidine, 7-(3-chloro-2-fluoroanilino)-3-(methyl, ethyl, isopropyl, 3-pentyl, benzyl)pyrazolo[4,3-d]pyrimidine, 7-(3-chloro-4-fluoroanilino)-3-(methyl, ethyl, isopropyl, 3-pentyl, benzyl)pyrazolo[4,3-d]pyrimidine, 7-(4-chloro-2-fluoroanilino)-3-(methyl, ethyl, isopropyl, 3-pentyl, benzyl)pyrazolo[4,3-d]pyrimidine, 7-(5-chloro-2-fluoroanilino)-3-(methyl, ethyl, isopropyl, 3-pentyl, benzyl)pyrazolo[4,3-d]pyrimidine, 7-(2-chloro-4-fluoro-5-methylanilino)-3-(methyl, ethyl, isopropyl, 3-pentyl, benzyl)pyrazolo[4,3-d]pyrimidine, 7-(5-chloro-4-fluoro-2-nitroanilino)-3-(methyl, ethyl, isopropyl, 3-pentyl, benzyl)pyrazolo[4,3-d]pyrimidine, 7-(5-chloro-2-hydroxyanilino)-3-(methyl, ethyl, isopropyl, 3-pentyl, benzyl)pyrazolo[4,3-d]pyrimidine, 7-(4-chloro-2-iodoanilino)-3-(methyl, ethyl, isopropyl, 3-pentyl, benzyl)pyrazolo[4,3-d]pyrimidine, 7-(2-chloro-4-iodoanilino)-3-(methyl, ethyl, isopropyl, 3-pentyl, benzyl)pyrazolo[4,3-d]pyrimidine, 7-(2-chloro-6-methylanilino)-3-(methyl, ethyl, isopropyl, 3-pentyl, benzyl)pyrazolo[4,3-d]pyrimidine, 7-(3-chloro-2-methylanilino)-3-(methyl, ethyl, isopropyl, 3-pentyl, benzyl)pyrazolo[4,3-d]pyrimidine, 7-[3-chloro-4-(trifluoromethoxy)anilino]-3-(methyl, ethyl, isopropyl, 3-pentyl, cyklopropyl, benzyl)pyrazolo[4,3-d]pyrimidine, 7-[4-chloro-2-(trifluoromethoxy)anilino]-3-(methyl, ethyl, isopropyl, 3-pentyl, cyklopropyl, benzyl)pyrazolo[4,3-d]pyrimidine, 7-(2-fluoroanilino)-3-(methyl, ethyl, isopropyl, 3-pentyl, cyklopropyl, benzyl)pyrazolo[4,3-d]pyrimidine, 7-(3-fluoroanilino)-3-(methyl, ethyl, isopropyl, 3-pentyl, cyklopropyl, benzyl)pyrazolo[4,3-d]pyrimidine, 7-(4-fluoroanilino)-3-(methyl, ethyl, isopropyl, 3-pentyl, cyklopropyl, benzyl)pyrazolo[4,3-d]pyrimidine, 7-(2-fluoro-4-iodoanilino)-3-(methyl, ethyl, isopropyl, 3-pentyl, benzyl)pyrazolo[4,3-d]pyrimidine, 7-(2-fluoro-5-nitroanilino)-3-(methyl, ethyl, isopropyl, 3-pentyl, cyklopropyl, benzyl)pyrazolo[4,3-d]pyrimidine, 7-(2-fluoro-4-methylanilino)-3-(methyl, ethyl, isopropyl, 3-pentyl, cyklopropyl, benzyl)pyrazolo[4,3-d]pyrimidine, 7-(3-fluoro-2-methylanilino)-3-(methyl, ethyl, isopropyl, 3-cyklopropyl, 3-pentyl, cyklopropyl, benzyl)pyrazolo[4,3-d]pyrimidine, 7-(3-fluoro-4-methylanilino)-3-(methyl, ethyl, isopropyl, 3-pentyl, cyklopropyl, benzyl)pyrazolo[4,3-d]pyrimidine, 7-(4-fluoro-2-methylanilino)-3-(methyl, ethyl, isopropyl, 3-pentyl, cyklopropyl, benzyl)pyrazolo[4,3-d]pyrimidine, 7-(3-fluoro-4-methylanilino)-3-(methyl, ethyl, isopropyl, 3-pentyl, cyklopropyl, benzyl)pyrazolo[4,3-d]pyrimidine, 7-(5-fluoro-2-methylanilino)-3-(methyl, ethyl, isopropyl, 3-pentyl, cyklopropyl, benzyl)pyrazolo[4,3-d]pyrimidine, 7-(4-fluoro-2-nitroanilino)-3-(methyl, ethyl, isopropyl, 3-pentyl, cyklopropyl, benzyl)pyrazolo[4,3-d]pyrimidine, 7-(4-fluoro-3-nitroanilino)-3-(methyl, ethyl, isopropyl, 3-pentyl, cyklopropyl, benzyl)pyrazolo[4,3-d]pyrimidine, 7-(2-jodoanilino)-3-(methyl, ethyl, isopropyl, 3-pentyl, cyklopropyl, benzyl)pyrazolo[4,3-d]pyrimidine, 7-[2-fluoro-4-(trifluoromethyl)anilino]-3-(methyl, ethyl, isopropyl, 3-pentyl, cyklopropyl, benzyl)pyrazolo[4,3-d]pyrimidine, 7-(4-iodo-2-methylanilino)-3-(methyl, ethyl, isopropyl, 3-pentyl, cyklopropyl, benzyl)pyrazolo[4,3-d]pyrimidine, 7-(2-methoxyanilino)-3-(methyl, ethyl, isopropyl, 3-pentyl, cyklopropyl, benzyl)pyrazolo[4,3-d]pyrimidine, 7-(3-methoxyanilino)-3-(methyl, ethyl, isopropyl, 3-pentyl, cyklopropyl, benzyl)pyrazolo[4,3-d]pyrimidine, 7-(4-methoxyanilino)-3-(methyl, ethyl, isopropyl, 3-pentyl, cyklopropyl, benzyl)pyrazolo[4,3-d]pyrimidine, 7-(2-methoxy-5-methylanilino)-3-(methyl, ethyl, isopropyl, 3-pentyl, cyklopropyl, benzyl)pyrazolo[4,3-d]pyrimidine, 7-(2-methoxy-6-methylanilino)-3-(methyl, ethyl, isopropyl, 3-pentyl, cyklopropyl, benzyl)pyrazolo[4,3-d]pyrimidine, 7-(4-methoxy-2-methylanilino)-3-(methyl, ethyl, isopropyl, 3-pentyl, cyklopropyl, benzyl)pyrazolo[4,3-d]pyrimidine, 7-(5-methoxy-2-methylanilino)-3-(methyl, ethyl, isopropyl, 3-pentyl, cyklopropyl, benzyl)pyrazolo[4,3-d]pyrimidine, 6-[4-methoxy-3-(trifluoromethyl)anilino]-3-(methyl, ethyl, isopropyl, 3-pentyl, cyklopropyl, benzyl)pyrazolo[4,3-d]pyrimidine, 7-(2-methylanilino)-3-(methyl, ethyl, isopropyl, 3-pentyl, cyklopropyl, benzyl)pyrazolo[4,3-d]pyrimidine, 7-(4-methylanilino)-3-(methyl, ethyl, isopropyl, 3-pentyl, cyklopropyl, benzyl)pyrazolo[4,3-d]pyrimidine, 7-(3-methylanilino)-3-(methyl, ethyl, isopropyl, 3-pentyl, cyklopropyl, benzyl)pyrazolo[4,3-d]pyrimidine, 7-[2-methyl-3-(trifluoromethoxy)anilino]-3-(methyl, ethyl, isopropyl, 3-pentyl, cyklopropyl, benzyl)pyrazolo[4,3-d]pyrimidine, 7-[2-methyl-4-(trifluoromethoxy)anilino]-3-(methyl, ethyl, isopropyl, 3-pentyl, cyklopropyl, benzyl)pyrazolo[4,3-d]pyrimidine, 7-[2-(methylthio)anilino]-3-(methyl, ethyl, isopropyl, 3-pentyl, cyklopropyl, benzyl)pyrazolo[4,3-d]pyrimidine, 7-[4-(methylthio)anilino]-3-(methyl, ethyl, isopropyl, 3-pentyl, cyklopropyl, benzyl)pyrazolo[4,3-d]pyrimidine, 7-(2-nitroanilino)-3-(methyl, ethyl, isopropyl, 3-pentyl, cyklopropyl, benzyl)pyrazolo[4,3-d]pyrimidine, 7-(3-nitroanilino)-3-(methyl, ethyl, isopropyl, 3-pentyl, cyklopropyl, benzyl)pyrazolo[4,3-d]pyrimidine, 7-(4-nitroanilino)-3-(methyl, ethyl, isopropyl, 3-pentyl, cyklopropyl, benzyl)pyrazolo[4,3-d]pyrimidine, 7-(2-nitro-4,5,6-trifluoroanilino)-3-(methyl, ethyl, isopropyl, 3-pentyl, cyklopropyl, benzyl)pyrazolo[4,3-d]pyrimidine, 7-[2-nitro-4-trifluoromethoxy)anilino]-3-(methyl, ethyl, isopropyl, 3-pentyl, cyklopropyl, benzyl)pyrazolo[4,3-d]pyrimidine, 7-(2-nitrotetrafluoroanilino)-3-(methyl, ethyl, isopropyl, 3-pentyl, cyklopropyl, benzyl)pyrazolo[4,3-d]pyrimidine, 7-(2,3,4,5,6-pentabromoanilino)-3-(methyl, ethyl, isopropyl, 3-pentyl, cyklopropyl, benzyl)pyrazolo[4,3-d]pyrimidine, 6-(2,3,4,5,6-pentafluoroanilino)-3-(methyl, ethyl, isopropyl, 3-pentyl, cyklopropyl, benzyl)pyrazolo[4,3-d]pyrimidine, 7-(2,3,4,5-tetrachloroanilino)-3-(methyl, ethyl, isopropyl, 3-pentyl, cyklopropyl, benzyl)pyrazolo[4,3-d]pyrimidine, 7-(2,3,5,6-tetrachloroanilino)-3-(methyl, ethyl, isopropyl, 3-pentyl, cyklopropyl, benzyl)pyrazolo[4,3-d]pyrimidine, 7-(4-(1,1,2,2-tetrafluoroethoxy)anilino)-3-(methyl, ethyl, isopropyl, 3-pentyl, cyklopropyl, benzyl)pyrazolo[4,3-d]pyrimidine, 7-(2,3,4,5,-tetrafluoroanilino)-3-(methyl, ethyl, isopropyl, 3-pentyl, cyklopropyl, benzyl)pyrazolo[4,3-d]pyrimidine, 7-(2,3,4,6,-tetrafluoroanilino)-3-(methyl, ethyl, isopropyl, 3-pentyl, cyklopropyl, benzyl)pyrazolo[4,3-d]pyrimidine, 7-(2,3,5,6,-tetrafluoroanilino)-3-(methyl, ethyl, isopropyl, 3-pentyl, cyklopropyl, benzyl)pyrazolo[4,3-d]pyrimidine, 7-[2,3,5,6-tetrafluoro-4-(trifluoromethyl)anilino]-3-(methyl, ethyl, isopropyl, 3-pentyl, cyklopropyl, benzyl)pyrazolo[4,3-d]pyrimidine, 7-(2,4,6-tribromoanilino)-3-(methyl, ethyl, isopropyl, 3-pentyl, cyklopropyl, benzyl)pyrazolo[4,3-d]pyrimidine, 7-(2,4,6-tribromo-3,5-dijodoanilino)-3-(methyl, ethyl, isopropyl, 3-pentyl, cyklopropyl, benzyl)pyrazolo[4,3-d]pyrimidine, 7-(2,3,4-trichloroanilino)-3-(methyl, ethyl, isopropyl, 3-pentyl, cyklopropyl, benzyl)pyrazolo[4,3-d]pyrimidine, 7-(2,4,5-trichloroanilino)-3-(methyl, ethyl, isopropyl, 3-pentyl, cyklopropyl, benzyl)pyrazolo[4,3-d]pyrimidine, 7-(2,4,6-trichloroanilino)-3-(methyl, ethyl, isopropyl, 3-pentyl, cyklopropyl, benzyl)pyrazolo[4,3-d]pyrimidine, 7-(2,4,5-trifluoroanilino)-3-(methyl, ethyl, isopropyl, 3-pentyl, cyklopropyl, benzyl)pyrazolo[4,3-d]pyrimidine, 7-(2,3,5-trifluoroanilino)-3-(methyl, ethyl, isopropyl, 3-pentyl, cyklopropyl, benzyl)pyrazolo[4,3-d]pyrimidine, 7-(2,3,6-trifluoroanilino)-3-(methyl, ethyl, isopropyl, 3-pentyl, cyklopropyl, benzyl)pyrazolo[4,3-d]pyrimidine, 7-(2,3,4-trifluoroanilino)-3-(methyl, ethyl, isopropyl, 3-pentyl, cyklopropyl, benzyl)pyrazolo[4,3-d]pyrimidine, 7-(2-trifluoromethoxyanilino)-3-(methyl, ethyl, isopropyl, 3-pentyl, cyklopropyl, benzyl)pyrazolo[4,3-d]pyrimidine, 7-(3-trifluoromethoxyanilino)-3-(methyl, ethyl, isopropyl, 3-pentyl, cyklopropyl, benzyl)pyrazolo[4,3-d]pyrimidine, 7-(4-trifluoromethoxyanilino)-3-(methyl, ethyl, isopropyl, 3-pentyl, cyklopropyl, benzyl)pyrazolo[4,3-d]pyrimidine, 7-(2,3,4-trifluoro-6-nitroanilino)-3-(methyl, ethyl, isopropyl, 3-pentyl, cyklopropyl, benzyl)pyrazolo[4,3-d]pyrimidine, 7-(2,4,5-trimethylanilino)-3-(methyl, ethyl, isopropyl, 3-pentyl, cyklopropyl, benzyl)pyrazolo[4,3-d]pyrimidine, 7-(2,4,6-trimethylanilino)-3-(methyl, ethyl, isopropyl, 3-pentyl, cyklopropyl, benzyl)pyrazolo[4,3-d]pyrimidine, 7-(3-chloro-4-carboxyanilino)-3-(methyl, ethyl, isopropyl, 3-pentyl, cyklopropyl, benzyl)pyrazolo[4,3-d]pyrimidine, 7-(3-carboxy-4-hydroxyanilino)-3-(methyl, ethyl, isopropyl, 3-pentyl, cyklopropyl, benzyl)pyrazolo[4,3-d]pyrimidine, 7-cyclohexylamino-3-(methyl, ethyl, isopropyl, 3-pentyl, cyklopropyl, benzyl)pyrazolo[4,3-d]pyrimidine, 7-cyclopentylamino-3-(methyl, ethyl, isopropyl, 3-pentyl, cyklopropyl, benzyl)pyrazolo[4,3-d]pyrimidine, 7-cyclobutylamino-3-(methyl, ethyl, isopropyl, 3-pentyl, cyklopropyl, benzyl)pyrazolo[4,3-d]pyrimidine, 7-allylamino-3-(methyl, ethyl, isopropyl, 3-pentyl, cyklopropyl, benzyl)pyrazolo[4,3-d]pyrimidine, 7-diallylamino-3-(methyl, ethyl, isopropyl, 3-pentyl, cyklopropyl, benzyl)pyrazolo[4,3-d]pyrimidine 7-isopentylamino-3-(methyl, ethyl, isopropyl, 3-pentyl, cyklopropyl, benzyl)pyrazolo[4,3-d]pyrimidine, 7-(3,3-dimethylallylamino)-3-(methyl, ethyl, isopropyl, 3-pentyl, cyklopropyl, benzyl)pyrazolo[4,3-d]pyrimidine, 7-(3-hydroxymethyl-3-methylallyl)amino-3-(methyl, ethyl, isopropyl, 3-pentyl, cyklopropyl, benzyl)pyrazolo[4,3-d]pyrimidine, 7-propargylamino-3-(methyl, ethyl, isopropyl, 3-pentyl, cyklopropyl, benzyl)pyrazolo[4,3-d]pyrimidine, 7-furfurylamino-3-(methyl, ethyl, isopropyl, 3-pentyl, cyklopropyl, benzyl)pyrazolo[4,3-d]pyrimidine, 7-(oxazol-4-yl)amino-3-(methyl, ethyl, isopropyl, 3-pentyl, cyclopropyl, benzyl)pyrazolo[4,3-d]pyrimidine, 7-(2-pyridylamino)-3-(methyl, ethyl, isopropyl, 3-pentyl, cyklopropyl, benzyl)pyrazolo[4,3-d]pyrimidine, 7-(3-pyridylamino)-3-(methyl, ethyl, isopropyl, 3-pentyl, cyklopropyl, benzyl)pyrazolo[4,3-d]pyrimidine, 7-(4-pyridylamino)-3-(methyl, ethyl, isopropyl, 3-pentyl, cyklopropyl, benzyl)pyrazolo[4,3-d]pyrimidine, 7-(4-morfolinyl)-3-(methyl, ethyl, 3-pentyl, benzyl)pyrazolo[4,3-d]pyrimidine, 7-(1-chinuklidinyl)-3-(methyl, isopropyl, 3-pentyl, benzyl)pyrazolo[4,3-d]pyrimidine, 7-(1-etyleniminyl)-3-(methyl, ethyl, 3-pentyl, benzyl)pyrazolo[4,3-d]pyrimidine, 7-(1-propyleniminyl)-3-(methyl, isopropyl, 3-pentyl, benzyl)pyrazolo[4,3-d]pyrimidine, 7-(1-pyrolidinyl)-3-(methyl, ethyl, isopropyl, 3-pentyl, benzyl)pyrazolo[4,3-d]pyrimidine, 7-(1-piperidinyl)-3-(methyl, ethyl, isopropyl, 3-pentyl, cyklopropyl, benzyl)pyrazolo[4,3-d]pyrimidine, 7-(1-piperazinyl)-3-(methyl, ethyl, isopropyl, 3-pentyl, cyklopropyl, benzyl)pyrazolo[4,3-d]pyrimidine, 7-pyrazol-3-(methyl, ethyl, isopropyl, 3-pentyl, cyklopropyl, benzyl)pyrazolo[4,3-d]pyrimidine, 7-imidazol-3-(methyl, ethyl, isopropyl, 3-pentyl, benzyl)pyrazolo[4,3-d]pyrimidine, 7-(1-imidazolinyl)-3-(methyl, ethyl, 3-pentyl, cyklopropyl, benzyl)pyrazolo[4,3-d]pyrimidine, 7-(1-pyrazolinyl)-3-(methyl, ethyl, isopropyl, 3-pentyl, cyklopropyl, benzyl)pyrazolo[4,3-d]pyrimidine.

The novel compounds of this invention per se or as intermediates in the preparation of novel compound having a wide variety of industrial utilities.

The compounds of the formula I and their pharmaceutically acceptable salts inhibit selectively the enzyme p34^{cdc2}/cyclin B kinase and related cdks (cdk1, cdk2, cdk5, cdk7, MAP kinases).

In another embodiment, this invention is a method for inhibiting cdks and cell proliferation and/or for inducing apoptosis in plants comprising administering an effective amount of the composition of claim 1 to the plant.

In still another embodiment, this invention is a composition useful for treating fungal infections (fungi) in humans, animals and plants.

Disubstituted pyrazolo[4,3-d]pyrimidine derivatives result in the acquisition of extremely high potency against viruses on the part of the defined compounds. An important aspect of the present invention is a method for inhibiting proliferation of a DNA virus dependent upon events associated with cell proliferation for replication. The DNA virus includes any of the retrovirus family. The effective amount is that sufficient to inhibit cellular CDK activity to extent impending viral replication.

In addition to other CDK1-related kinases, this kinase controls certain steps of cell division cycles, in particular the transition from G₁ phase into the S phase and in particular the transition from the G₂ phase into the M-phase. Out the basis of this findings, it can be expected that the compounds of the formula I and their acceptable salts can be used as antimitotic compounds and for treatment of proliferative diseases.

In addition to therapeutic applications it will be apparent the subject compounds can be used as a cell culture additive for controlling proliferative and/or differentiation states of cells in vitro, for instance, by controlling the level of activation of a CDK. By preventing the activation of a Go/G₁ CDK, the subject inhibitors can prevent mitotic progression and hence provide a means for ensuring an adequately restrictive environment in order to maintain cells at various stages of differentiations, and can be employed, for instance, in cell cultures designed to test the specific activities of trophic factors. Other tissue culture systems which require maintenance of differentiation will be readily apparent to those skilled in the art.

It is likely that inhibition by the compounds, of the invention of the catalytic activity of cyclin-dependent kinases in mediated by interaction of the compounds at the ATP-binding site of the enzyme. Such compounds are particularly desirable for reducing excessive cell growth, since they allow inhibition of the kinase activity regardless of the cause underlying the excessive kinase activity leading to excessive cell proliferation. Thus, the compounds of the invention are active in situations in which the excessive kinase activity results from the kinase being a mutated hyperactive, form of the kinase and situations in which the kinase is present at excessive levels. Such compounds can also block excessive kinase activity in situations in which the cyclin regulating the kinase is present at excessive levels or its binding to the kinase is enhanced. Furthermore, compounds which block kinase activity by interacting with the ATP binding site of the enzyme are also useful for inhibiting kinase activity in situations in which a natural inhibitor of cyclin-kinase complexes is mutated.

It will also be apparent that differential screening assays can be used to select for those compounds of the present invention with specificity for CDK enzymes. Thus, compounds, which act specifically on eukaryotic pathogens, e.g., are anti-fungal or anti-parasitic agents, can be selected from the subject of the inhibitors.

By way of illustration, the assays described in the art can be used to screen for agents which may ultimately be useful for inhibiting at leas one fungus implicated in such mycosis as aspergillosis, blastomycosis, chromoblastomycosis, coccidiomycosis, conidiosporosis, actinomycosis, penicilliosis, monoliasis, or sporotrichosis. For example, if the mycotic infection to which treatment is desired is aspergillosis, an assay as described above or in the appended examples can comprise comparing the relative effectiveness of a test compound on inhibiting a plant CDK enzyme with its effectiveness towards a CDK enzyme from yeast. Likewise, the differential screening assays can be used to identify anti-fungal agents which may have value in the treatment of aspergillosis by making use of the CDK genes cloned from yeast such as Aspergillus fumigatus, Aspergillus flavus, Aspergillus niger, Aspergillus nidulans, or Aspergillus terreus.

In yer another embodiment, certain of the subject CDK inhibitors can be selected on the basis of inhibitory specificity for plant CDK s relative to the mammalian enzyme. For example, a plant CDK can be sidposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

### Processes for Preparation

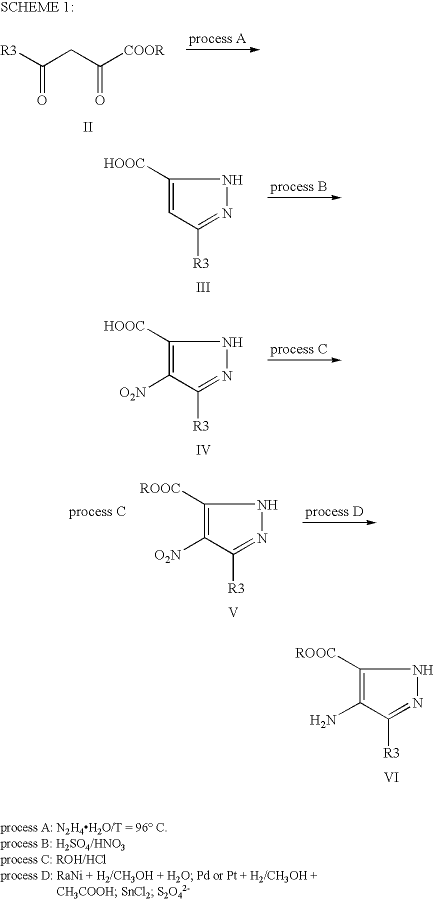

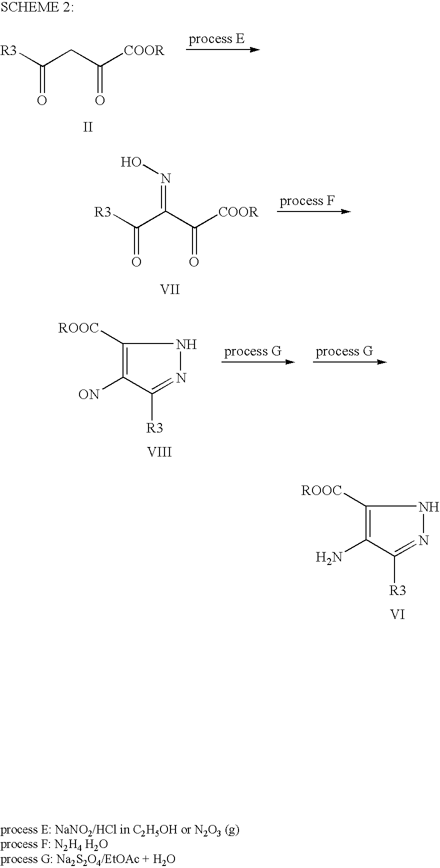

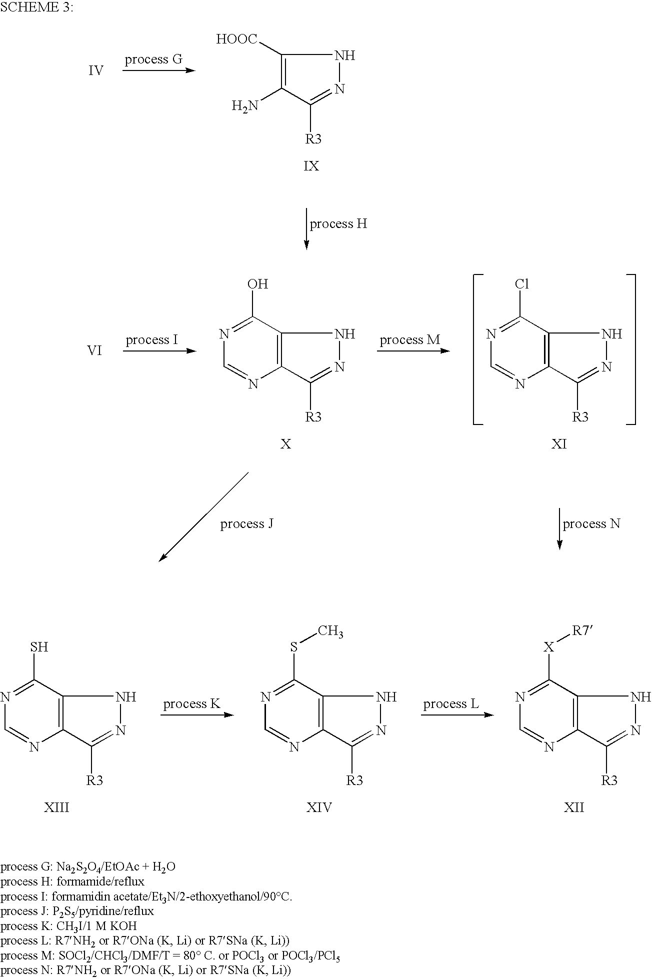

In one approach the 3-isopropyl-7-substituted pyrazolo[4,3-d]pyrimidine of the formula I, wherein R7 substituents are defined above for a compound of the formula I, are prepared by reaction of 7-chloro-3-isopropylpyrazolo[4,3-d]pyrimidine with appropriate nucleophile as 1)amine (ammonium hydroxide, hydrazine, hydroxylamine, benzylamine; 2-, 3-, 4-hydroxybenzylamine; dihydroxybenzylamine; 3-chloroaniline, etc.) or 2) lithium (natrium, kalium) salt of alcohole or mercaptane. Preferably, the appropriate nucleophil reactant may be R7′-X—Y, wherein R7′-X— is as defined in claim 1 and Y is H. A nucleophil is reagent is able to attack a place in molecule with absence of electrons. An appropriate alkylating agent is a reagent which is source of carbo cations which attack a place in a molecule with excess of electrons—preferentially free electron couples, usually oxygen, nitrogen and sulfur, such as R7′-Z, wherein R7′ is as defined in claim 1 and Z is selected from halogen, toluensulfonate, and mesylate. 7-Chloro-3-isopropylpyrazolo[4,3-d]pyrimidine is dissolved in chloroform and appropriate R^{7′}—NH₂ or R^{7′}—O(Li, Na, K), or R^{7′}—S(Li, Na, K) (5-20 eq.) was added. After heating for several hours, the reaction mixture is cooled and the 7-substituted-3-isopropylpyrazolo[4,3-d]pyrimidine is obtained.

In another approach the 3-ethyl substituted pyrazolo[4,3-d]pyrimidine of the formula I, wherein R7 substituents are defined above for a compound of the formula L are prepared by reaction of 7-chloro-3-ethylpyrazolo[4,3-d]pyrimidine with appropriate nucleophile as 1)amine (ammonium hydroxide, hydrazine, hydroxylamine, benzylamine; 2-, 3-, 4-hydroxybenzylamine; dihydroxybenzylamine; 3-chloroaniline, etc.) or 2) lithium(natrium, kalium) salt of alcohole or mercaptane. 7-Chloro-3-isopropylpyrazolo[4,3-d]pyrimidine is dissolved in chloroform and appropriate R^{7′}—NH₂ or R^{7′}—O(Li, Na, K), or R^{7′}—S(Li, Na, K) (5-20 eq.) was added. After heating for several hours, the reaction mixture is cooled and the 7-substituted-3-ethylpyrazolo[4,3-d]pyrimidine is obtained.

In yet another approach the 3-methyl substituted pyrazolo[4,3-d]pyrimidine of the formula I, wherein R7 substituents are defined above for a compound of the formula I, are prepared by reaction of 7-chloro-3-methylpyrazolo[4,3-d]pyrimidine with appropriate nucleophile as 1)amine (ammonium hydroxide, hydrazine, hydroxylamine, benzylamine; 2-,3-,4hydroxybenzylamine; dihydroxybenzylamine; 3-chloroaniline, etc.) or 2) lithium(natrium, kalium) salt of alkohole or mercaptane. 7-Chloro-3-isopropylpyrazolo[4,3-d]pyrimidine is dissolved in chloroform and appropriate R^{7′}—NH₂ or R^{7′}—O(Li, Na, K), or R^{7′}—S(Li, Na,K) (5-20 eq.) was added. After heating for several hours, the reaction mixture is cooled and the 7-substituted-3-methylpyrazolo[4,3-d]pyrimidine is obtained.

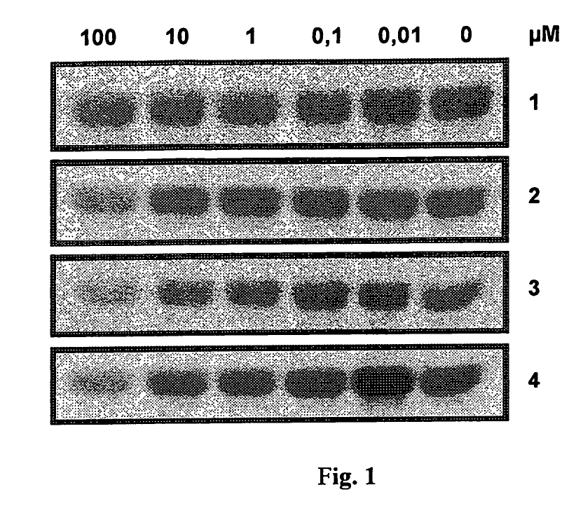

Further, FIG. 1 shows a diagram displaying the specific inhibition of plant cdc2 kinase activity in plant cells. Enzyme activity bound to p13^{suc1}-agarose was measured by phosphorylation of histone H1 substrate protein in the presence of various concentrations of (1) 7-furfurylamino-3-methylpyrazolo[4,3-d]pyrimidine, (2) 7-benzylamino-3-isopropylpyrazolo[4,3-d]pyrimidine, (3) 7-(3-chloroanilino)-3-isopropylpyrazolo[4,3-d]pyrimidine, (4) 7-(3-hydroxybenzyl)amino-3-isopropylpyrazolo[4,3-d]pyrimidine.

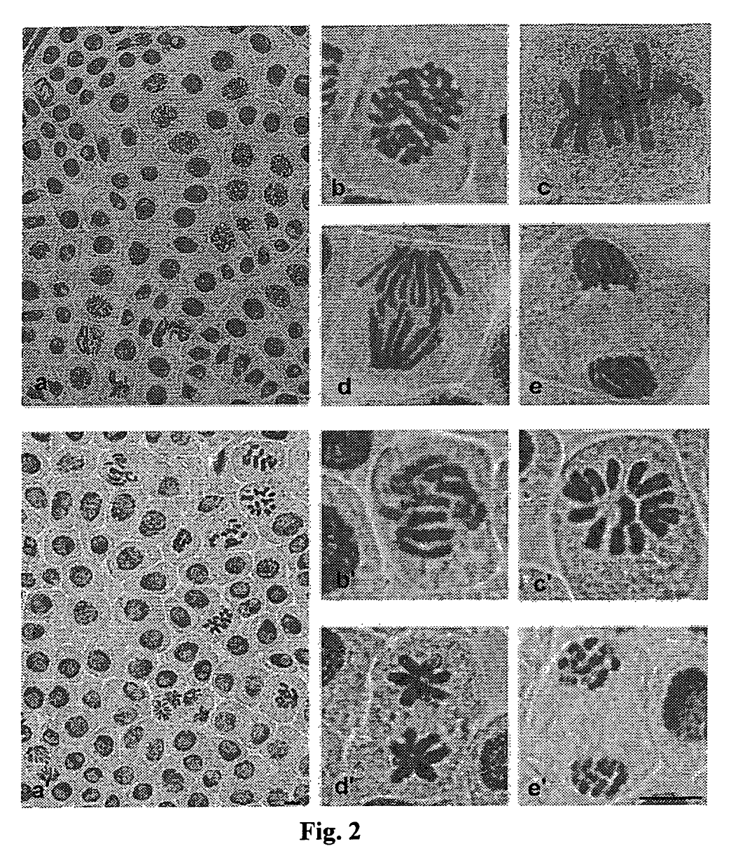

FIG. 2 shows pictures of the induction of aberrant mitosis in root meristem cells of V. faba after the treatment with 200 mM A2.16.32, wherein

pictures a-e—control cells

pictures a′-e′—cells treated with 200 mM A2.16.32 for 12 hr.

picture a—lower magnification showing frequency od mitosis (a), and aberrant mitosis (a′). b,b′—prophase, c,c′—metaphase, d,d′—anaphase, e,e′—telophase.

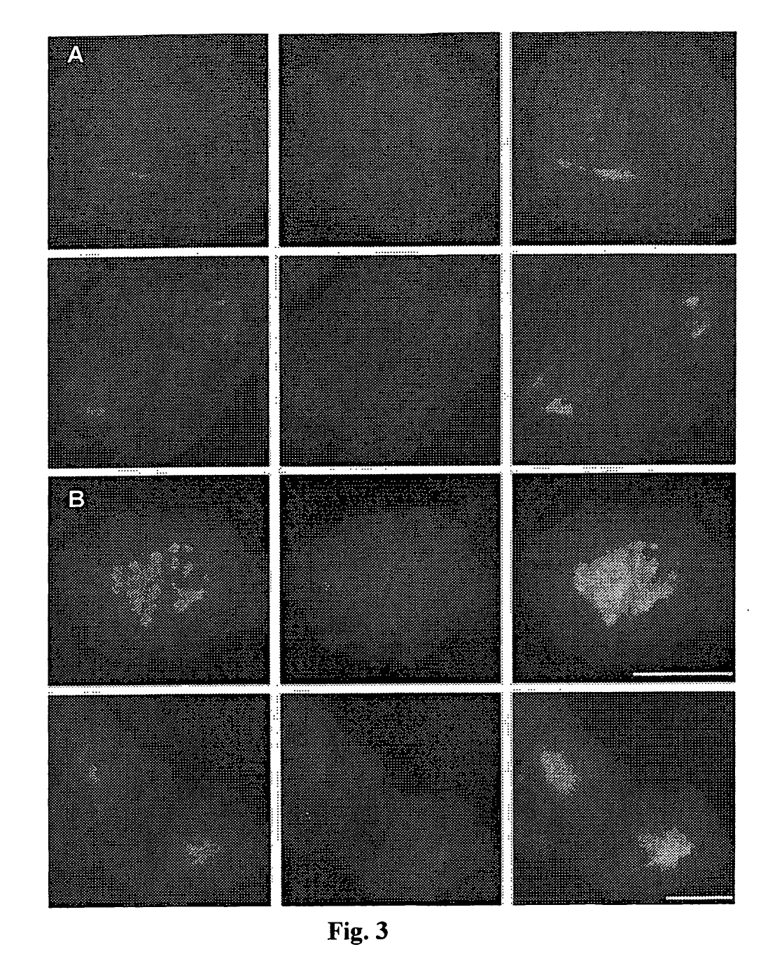

FIG. 3 shows immunofluorescence visualization of microtubules in control (A) and treated (B) root meristem cells of V.faba. Green—FITC immunolabelling for alfa-tubulin (left column), red—immunolabelling for gamma-tubulin (middle column), blue—immunolabelling for DNA labelling with DAPI (right column).

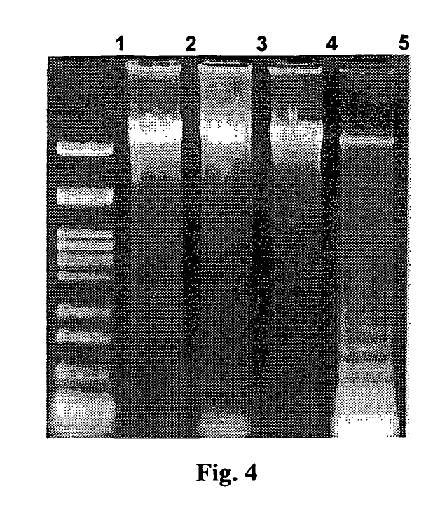

FIG. 4 shows pictures of the electrophoretic detection of double strand DNA breaks (marker of apoptosis) after the treatment of root meristem cells of Vicia faba with bohemine (200 μM) wherein the slots are

1. molecular weight markers

2. control I—DNA after 10 h incubation without the drug

3. DNA after 10 h incubation with bohemine

4. control II—DNA after 44 hod inkubation without the drug and

5. DNA pafter 44 hod incubation with bohemine.

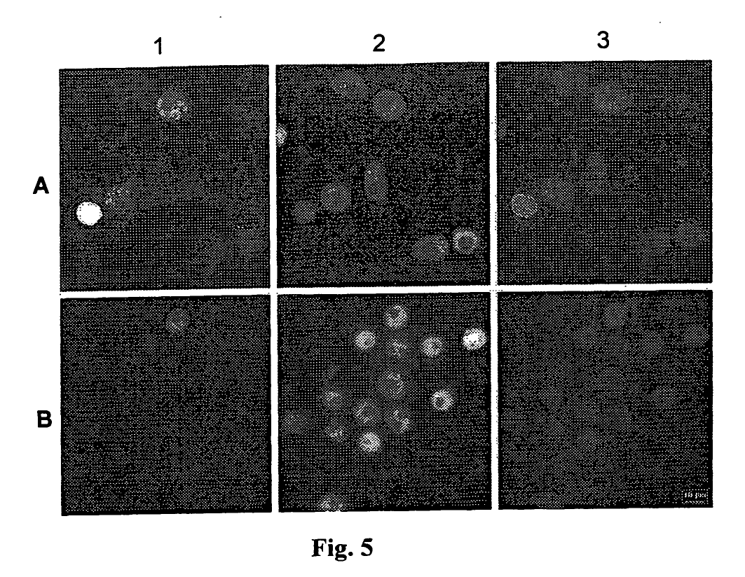

FIG. 5 shows immunofluorescence detection of DNA double strand breaks in root meristem cells of V. faba treated with bohemine.
A. Root meristem cells of V. faba, treated with bohemine (200 mM ). 1^{st} line
B. Control cells. 2nd. line
1. column —FITC labelling of DNA breaks.
2. column—DAPI labelling for chromatin
3. column—merged images of FITC and DAPI

### THE FOLLOWING EXAMPLES SERVE TO ILLUSTRATE THE INVENTION WITHOUT LIMITING THE SCOPE THEREOF

The starting material for the compounds of the formula I is available from commercial sources (Sigma, Aldrich, Fluka, etc.). Melting points were determined on a Koffler block and are uncorrected. Evaporations were carried out on a rotary evaporator under vacuum at temperatures below 80° C. The ¹H NMR spectra (c, ppm; J, Hz) were measured on Varian VXR-400 (400 MHz) or on Varian Unity 300 (400 MHz) instruments. All spectra were obtained at 25° C. using tetramethylsilane as on a internal standard. Electron impact mass spectra m/z (rel. %, composition, deviation) were measured on a VG 7070E spectrometer (70 eV, 200° C., direct inlet). Quadrupole mass spectra were measured on a Micromass ZMD detector with electrospray ionization. Merck silica gel Kieselgel 60 (230-400 mesh) was used for column chromatography. All compounds gave satisfactory elemental analyses (±0.4%).

### Example 1

### methyl 2,4-dioxo-5-methylhexenoate (II)

Prepared according to: E. Royals: J. Am. Chem. Soc. 67, 1508 (1945)

### Example 2

### 5-isopropylpyrazol-3-carboxylic acid (III)

A solution of methyl 2,4-dioxo-5-methylhexenoate II (31g; 180 mmol) and N₂H₄.H₂O (13 mL, 180 mmol) in ethanol (120 mL ) was refluxed for 2 hours. The mixture was poured into H₂O and extracted with chloroform. The chloroform extract was concentrated under reduced pressure to give a yellow liquid. The liquid and aq. 3 M NaOH (120 mL) was stirred overnight at room temperature. The acidification of the yellow solution to pH=2 with conc. HCl affords crystals. Crystals were filtered off and washed with ice-water. Yield 68%, mp=136-140° C.

¹H NMR(300 MHz, MeOD): 1.14d(6H, 7.1 Hz), 3.02sept.(1H, 7.1 Hz), 6.59s(1H).; CHN required: C=54.54%; H=6.54%; N=18.17%; found: C=54.58%; H=6.38%; N=18.12%.

### Example 3

### 5-isopropyl-4-nitropyrazole-3-carboxylic acid (IV)

To an ice-cooled and stirred solution of 2.9 g (18.8 mmol) 5-isopropylpyrazol-3-carboxylic acid III in fuming sulphuric acid 1 mL (65%,), sulphuric acid 7.6 mL (100%) and the nitric acid 3 mL (65%) was added portionwise. The stirring was continued for 1 h at room temperature and then another 3 h at 104° C. temperature and then poured into ice-water. The white precipitate of product was filtered and crystallized from water; (yield 76%); mp=139-142° C.; ¹H NMR(300 MHz, DMSO): 1.22d(6H, 7.1 Hz), 2.94sept(1H, 7.1 Hz), 3.33s(1H), 6.45bs(1H); CHN required: C=42.02%; H=4.56%; N=21.09%; found: C=42.41%; H=4.49%; N=21.01%.

### Example 4

### Methyl 5-isopropyl-4-nitropyrazol-3-carboxylate (V)

5-Isopropyl-4-nitropyrazole-3-carboxylic acid was added to a 4.5M solution of HCl in absolute methanol. The reaction mixture was heated at 60° C. for 7 hours and then was evaporated to dryness. The title compound was crystallised from ethyl acetate; yield 91%; mp=78-80° C. ¹H NMR (300 MHz, CDCl₃): 1.39d(6H, J=7.1 Hz); 3.64sept(1- J=7.1 Hz), 3.98s(3H). CHN required: C=45.07%; H=5.20%; N=19.70% found: C=45.21%; H=5.23%; N=19.65%.

### Example 5

### Methyl 4amino-5 isopropylpyrazol-3-carboxylate (VI)

Mode A

To a solution of methyl 5-isopropyl-4-nitropyrazol-3-carboxylate (4.34g, 24 mmol) in 20 mL ethanol and 5 mL water was added 1 g RaNi (an activity W5). The mixture was stirred under hydrogen atmosphere (760 Torr) for 12 hours. The RaNi was filtered off and the filtrate was concenrated in vacuo. The residue crystals were washed with cooled ethylacetate; yield 95%; mp=122-123° C. MS(EI, 70 eV, direct inlet): 183(88; C₈H₁₃N₃O₂.⁺.; −1.0), 168(59), 152(3), 136(100), 108(8), 80(16), 68(20). ¹H NMR(400 MHz, CDCl₃): 1.31d(6H; J=6.9 Hz), 2.93sept(1H; J=6.9 Hz), 3.9s(3H). IR (KBr, cm⁻¹): 3399, 3296, 1710, 1626, 1584, 1302.

Mode B

To a solution of methyl 4-nitropyrazol-3-carboxylate V (7.34g, 34.4 mmol) in 36 mL n-propanole, 6 mL water and 5.6 mL 10 M HCl was added 0.55 g PtO₂. The mixture was stirred under hydrogen atmosphere (760 Torr) for 9 hours. The reaction mixture was filtered and the filtrate was concentrated to dryness in vacuo. The desired amine was liberated by treatment of aq. ammonia during extraction into chloroform. The product crystallised after evaporation; yield 95%; mp=122-123° C.

### Example 6

### 7-hydroxy-3-isopropylpyrazolo[4,3-d]pyrimidine (X)

A mixture of aminoester VI (1.5 g, 8.42 mmol), formamidine acetate (2.47 g, 24 mmol) and triethylamine (5.25 mL ) in 32 mL of 2-ethoxyetanol was heated for 2 hours at 90° C. under argon atmosphere. The excess of triethylamine was evaporated from cellosolve solution in vacuo, the crystallised product was filtered off and washed with chloroform. An analytical sample was obtained by recrystallisation from ethanol. Yield 96%; mp=302-304° C. ¹H-NMR (300 MHz, CD₃OD): 1.41d(6H, J=7.15 Hz), 3.40sept(1H, J=7.15 Hz), 7.82s(1H). ¹³C-NMR (400 MHz, DMSO-d₆+AcOD): 21.912, 25.985, 141.85, 172.17. CHN required: C=53.92%; H=5.66%; N=31.44; found: C=53.20%; H=5.58%; N=31.39%. MS (EI): 178(35; C₈H₁₀N₄O.⁺); 177(8) 163(25;C₇H₇N₄O.⁺); 150(18;C₆H₆N₄O.⁺); 54(18); 53(10); 41(11); 28(38); 27(11).

### Example 7

### [7-chloro-3-isopropylpyrazolo[4,3-d]pyrimidine (XI)]

7-Hydroxy-3-isopropylpyrazolo[4,3-d]pyrimidine X (200 mg, 1.122 mmol) was dissolved in the mixture of 0.81 mL (11 mmol) of thionyl chloride, 0.12 mL (1.56 mmol) of dimethylformamide and 5 mL of chloroform. This mixture was heated under reflux for 3 hours. The solution was evaporated to dryness in vacuo and the residue was dissolved in chloroform. This solution was extracted twice with a small portions of water and combined chloroform extract was dried over Na₂SO₄. This compound VIII was not isolated and was used immediately as chloroform solution in following reaction step.

### Example 8

### 7-benzylamino-3-isopropylpyrazolo[4,3-d]pyrimidine XIIa

To chloroform solution of XI (prepared in the subsequent step from 200 mg X) was added 3 mL of benzylamine. This mixture was stirred at room temperature 10 minutes and then evaporated to dryness in vacuo. The crude product was purified by column chromatography on silica gel, the mixture of chloroform/methanol (98.5 /1.5) was used as mobile phase. Yield 82%; mp=153-154° C. ¹H-NMR (400 MHz, CDCl₃): 1.40d(6H, J=7.02 Hz); 3.41sept(1H, J=7.02 Hz); 4.80s(2H), 7.25m(5H), 6.57s (1H), 8.4s (1H). CHN required: C=67.39%; H=6.41%; N=26.20; found: C=67.33%; H=6.43%; N=26.24%. MS (EI): 267(62;C₈H₁₀N₄O.⁺); 266(18) 252(44); 239(6); 106(49); 91(100); 65(21); 43(14); 41(16).

### Example 9

### 7-(2-hydroxybenzyl)amino-3-isopropylpyrazolo[4,3-d]pyrimidine XIIb

To chloroform solution of XI (prepared in the subsequent step from 200 mg X) was added 1.31 mL (7.7 mmol) N-ethyldiisopropylamine, 3 mL EtOH and 500 mg (4.06 mmol) 2-hydroxybenzylamine. This mixture was heated one hour at 60° C. and then was evaporated to dryness in vacuo. The crude product was purified by chromatography on silica gel in the mixture of chloroform/methanol/AcOH (20:0.4:0.1). Yield 40%; mp=214-217° C. ¹H-NMR (400 MHz, DMSO-d₆): 1.36d(6H, J=6.96 Hz); 3.28sept(1H, J=6.96 Hz); 4.65s(2H), 6.78-7.26m(4H), 8.21s (1H). CHN required: C=63.59%; H=6.05%; N=24.72; found: C=63.39%; H=6.07%; N=24.62%. MS (ES): [M+H]⁺=274.3 (100).

### Example 10

### 7-(3-hydroxybenzylamino)-3-isopropylpyrazolo[4,3-d]pyrimidine XIIc

To chloroform solution of XI (prepared in the subsequent step from 200 mg X) was added 1.5 mL (8.8 mmol) N-ethyldiisopropylamine, 5 mL EtOH and 500 mg (4.06 mmol) 3-hydroxybenzylamine. This mixture was heated one hour at 60° C. and then was evaporated to dryness in vacuo. The crude product was purified by column chromatography on silica gel in the mixture of chloroform/methanol/AcOH (20:0.6:0.1). Yield 48%; mp=220-221° C. ¹H-NMR (300 MHz, DMSO-d₆): 1.38d(6H, J=7.14 Hz); 3.26sept(1H, J=7.14 Hz); 4.68s(2H), 6.62-7.17m(3H), 8.22s (1H); 9.31s(1H). MS (ES): [M+H]⁺=274.3 (100).

### Example 11

### 7-(4-hydroxybenzyl)amino-3-isopropylpyrazolo[4,3-d]pyrimidine XIId

To chloroform solution of XI (prepared in the subsequent step from 200 mg X) was added 1.5 mL (8.8 mmol) N-ethyldiisopropylamine, 5 mL EtOH and 500 mg (4.06 mmol) 4-hydroxybenzylamine. This mixture was heated one hour at 60° C. and then was evaporated to dryness in vacuo. The crude product was purified by column chromatography on silica gel in the mixture of chloroform/methanol/AcOH (20:1:0.1). Yield 49%; mp=234-236° C. ¹H-NMR (300 MHz, DMSO-d₆): 1.28d(6H, J=6.59 Hz); 3.58sept(1H, J=6.59 Hz); 4.60s(2H), 6.73-7.21m(4H), 8.20s (1H). MS (ES): [M+H]⁺=274.3 (100).

### Example 12

### 7-(3-chloroanilino)-3-isopropylpyrazolo[4,3-d]pyrimidine XIIe

To chloroform solution of XI (prepared in the subsequent step from 200 mg X) was added 1.20 ml (11.2 mmol) 3-chloroaniline. This mixture was heated one hour at 60° C., then was cooled at room temperature and crystals were precipitated. These colourless crystals were washed with ether; the analytical sample was obtained by recrystallisation from mixture ethanol-ether. Yield 58%; mp=213-216° C. ¹H-NMR (400 MHz, DMSO-d₆): 1.40d(6H, J=6.94 Hz); 3.48sept(1H, J=6.94 Hz); 7.30dd(1H), 7.49dd(1H), 7.86dd(1H), 8.20s(1H), 8.78s (1H). CHN(C₁₄H₁₄N₅Cl.HCl.H₂O) required: C=49.20%; H=5.01%; N=20.48%; Cl=20.48%; found: C=49.43%; H=5.09%; N=20.13%; Cl=20.58%. MS (ES): [M+H]⁺=288.5 (100), 290.5 (33).

### Example 13

### 7-(isopent-2-en-(1-yl)amino)-3-isopropylpyrazolo[4,3-d]pyrimidine XIIf

To chloroform solution of XI (prepared in the subsequent step from 200 mg X) was added 3.5 mL (20 mmol) N-ethyldiisopropylamine, 3 mL EtOH and 620 mg (4.7 mmol) isopent-2-en-1-(yl)amino hydrochloride. This mixture was stirred 12 hours at room temperature and then was evaporated to dryness in vacuo. The crude product was purified by column chromatography on silica gel in the mixture of chloroform/methanol/aq. NH₄OH (98:2:1). Yield 48%; syrupy. ¹H-NMR (400MHz, CDCl₃): 1.45d(6H, J=6.96 Hz); 1.65d(1H, J=1.28 Hz), 3.47sept(1H, J=6.96 Hz); 4.18d(2H, J=1.28 Hz), 5.25m(1H, J=1.28 Hz), 6.25s(1H), 8.22s (1H). COSY[1.45d(6H, J=6.96 Hz); 3.47sept(1H, J=6.96 Hz)], COSY[1.65d(6H, J=1.28 Hz); 4.18d(2H, J=1.28 Hz); 5.25m(1H, J=1.28 Hz)], COSY[4.18d(2H); 6.25s(1H)]. MS (ES): [M+H]⁺=246.5 (100).

### Example 14

### 7-furfurylamino-3-isopropylpyrazolo[4,3-d]pyrimidine XIIg

To the chloroform solution of XI (prepared in the subsequent step from 150 mg X) was added 2 mL (0.205 mol) furfurylamine. This mixture was stirred 1 hour at 50° C. and then was evaporated to dryness in vacuo. The crude product was purified by column chromatography on silica gel in the mixture of chloroform/methanol/aq. NH₄OH (98:2:0.2). Yield 43%; mp=179-182° C. ¹H-NMR (500 MHz, MeOD): 1.422d (6H, J=7.0 Hz); 3.455sept. (1H, J=7.0 Hz); 4.802s (2H); 6.373s (2H); 7.468s (1H); 8.273s (1H). MS (ES): [M+H]⁺=258.3 (100).

### Example 15

### 7-pentylamino-3-isopropylpyrazolo[4,3-d]pyrimidine XIIh

To the chloroform solution of XI (prepared in the subsequent step from 150 mg X) was added 0.29 mL (2.53 mmol) 3-pentylamine. This mixture was stirred 1 hour at 50° C. and then was evaporated to dryness in vacuo. The crude product was purified by column chromatography on silica gel. in the mixture of chloroform/methanol (99:1). Yield 25%; mp=73-75° C. ¹H-NMR (500 MHz, MeOD): 0.933t (3H, J=7.0 Hz); 1.374bs (2H); 1.388bs (2H); 1.418d (6H, J=6.9 Hz); 1.715pent. (2H, J=7.0 Hz), 3.447hept.(1H, J=6.9 Hz); 3.583t (2H, J=7.0 Hz); 8.207s (1H). MS (ES): [M+H]⁺=248.2 (100).

### Example 16

### 7-(2-bromobenzyl)amino-3-isopropylpyrazolo[4,3-d]pyrimidine XIIk

To chloroform solution of XI (prepared in the subsequent step from 152 mg X, 0.855 mmol) was added 0.44 mL (2.60 mmol) N-ethyldiisopropylamine, 2 ml methanol and 343 mg (1.54 mmol) 2-bromobenzylamine hydrochloride. This mixture was heated two hours at 60° C. and then was evaporated to dryness in vacuo. The crude product was purified by column chromatography on silica gel in the mixture of chloroform/methanol (98.5:1.5) crystallization from Et₂O. Yield 42%; mp=194-196° C. ¹H-NMR (300 MHz, CH₃OD): 1.44d(6H, J=6.9 Hz); 3.43hept(1H, J=6.9 Hz); 4.89s(2H); 7.20t(1H, J=7.1 Hz); 7.32t(1H, J=7.1 Hz); 7.45bs(1H); 7.62d(1H, J=7.1 Hz); 8.24s (1H). MS (ES): [M+H]⁺=246.2 (95), 248.2 (100).

### Example 17

### 7-(4-methoxybenzyl)amino-3-isopropylpyrazolo[4,3d]pyrimidine XIIl

To chloroform solution of XI (prepared in the subsequent step from 152 mg X, 0.855 mmol) was added 0.34 mL (2.60 mmol) 4-methoxybenzylamine. This mixture was heated one hour at 52° C. and then was evaporated to dryness in vacuo. The crude product was purified by column chromatography on silica gel in the mixture of chloroform/methanol (98:2), crystallization from Et₂O. Yield 42%; mp=143-144° C. ¹H-NMR (300 MHz, CDCl₃): 1.36d(6H, J=6.9 Hz); 3.46sept. (1H, J=6.9 Hz); 3.69s (3H), 4.86bs (2H); 6.72d (2H, J=8.8 Hz); 7.30d (2H, J=8.8 Hz); 8.34s (1H). MS (ES): [M+H]⁺=298.3 (100).

### Example 18

### 7-(3-hydroxy-4-methoxybenzyl)amino-3-isopropylpyrazolo[4,3d]pyrimidine XIIm

To chloroform solution of XI (prepared in the subsequent step from 152 mg X, 0.855 mmol) was added 0.44 mL (2.60 mmol) N-ethyldiisopropylamine, 7 mL methanol and 236 mg (1.54 mmol) 3-hydroxy-4-methoxybenzylamine. This mixture was heated one hour at 52° C. and then was evaporated to dryness in vacuo. The crude product was purified by column chromatography on silica gel in the mixture of chloroform/methanol/NH₄OH (93.5:6.5:0.1) crystallization from chloroform/Et₂O. Yield 62%; mp=197-199° C. ¹H-NMR (300 MHz, CH₃OD): 1.43d(6H, J=7.15 Hz); 4.45hept(1H, J=7.15 Hz); 3.83s(3H); 4.67s(2H), 6.82-6.90m (3H), 8.24s (1H). MS (ES): [M+H]⁺=314.3 (100).

TABLE 1
Compounds Prepared by the Method of Examples 8-18

| PYRAZOLO[4,3-d]PYRIMIDINE | | | CHN | MS | |
|---|---|---|---|---|---|
| SUBSTITUENT | | | ANALYSES | ANALYSES-ZMD | |
| | C7 | C3 | [%] | [M − H]⁻ a) | M + H]⁺ b) |
| 1 | 3-chloroanilino | methyl | C = 55.50; H = 3.88 | | 260.1 |
| | | | N = 26.97; Cl = 13.65 | | 262.1 |
| 2 | anilino | methyl | C = 63.99; H = 4.92 | | 226.1 |
| | | | N = 31.09 | | |
| 3 | 4-bromoanilino | methyl | C = 47.39; H = 3.31 | 303.0 | 304.0 |
| | | | N = 23.03; Br = 26.27 | 305.0 | 306.0 |
| 4 | 4-chloroanilino | methyl | C = 55.50; H = 3.88 | | 260.1 |
| | | | N = 26.97; Cl = 13.65 | | 262.1 |
| 5 | 2-hydroxybenzylamino | methyl | C = 61.17; H = 5.13 | 254.1 | 256.1 |
| | | | N = 27.43 | | |
| 6 | 3-hydroxybenzylamino | methyl | C = 61.17; H = 5.13 | 254.1 | 256.1 |
| | | | N = 27.43 | | |
| 7 | 4-hydroxybenzylamino | methyl | C = 61.17; H = 5.13 | 254.1 | 256.1 |
| | | | N = 27.43 | | |
| 8 | 2-methylbenzylamino | methyl | C = 66.38; H = 5.79 | | 254.1 |
| | | | N = 27.65 | | |
| 9 | 3-methylbenzylamino | methyl | C = 66.38; H = 5.79 | | 254.1 |
| | | | N = 27.65 | | |
| 10 | 4-methoxybenzylamino | methyl | C = 62.44; H = 5.61 | | 270.0 |
| | | | N = 26.00 | | |
| 11 | 2-chlorobenzylamino | methyl | C = 37.04; H = 4.42 | | 274.1 |
| | | | N = 25.59; Cl = 12.95 | | 276.1 |
| 12 | 2-bromobenzylamino | methyl | C = 49.08; H = 3.80 | | 319.0 |
| | | | N = 22.01; Br = 25.11 | | 321.0 |
| 13 | 3-chlorobenzylamino | methyl | C = 57.04; H = 4.42 | | 274.1 |
| | | | N = 25.59; Cl = 12.95 | | 276.1 |
| 14 | 3-hydroxy-4-methoxybenzylamino | methyl | C = 58.94; H = 5.30 | | 286.0 |
| | | | N = 24.55 | | |
| 15 | furfurylamino | methyl | C = 57.63; H = 4.84 | | 230.1 |
| | | | N = 30.55 | | |
| 16 | allylamino | methyl | C = 57.13; H = 5.86 | | 190.1 |
| | | | N = 37.01 | | |
| 17 | cyclohexylamino | methyl | C = 62.31; H = 7.41 | | 232.2 |
| | | | N = 30.28 | | |
| 18 | 1,4-(trans)-cyclohexyldiamino | methyl | C = 58.52; H = 7.37 | | 247.0 |
| | | | N = 34.12 | | |
| 19 | 1,2-(cis)-cyclohexyldiamino | methyl | C = 58.52; H = 7.37 | | 247.0 |
| | | | N = 34.12 | | |
| 20 | cyclopentylamino | methyl | C = 60.81; H = 6.96 | | 218.1 |
| | | | N = 32.23 | | |
| 21 | cyclobutylamino | methyl | C = 59.10; H = 6.45 | | 204.1 |
| | | | N = 34.46 | | |
| 22 | (isopent-2-en-1-yl)amino | methyl | C = 60.81; H = 6.96 | | 204.1 |
| | | | N = 32.23 | | |
| 23 | pentylamino | methyl | C = 60.23; H = 7.81 | | 220.0 |
| | | | N = 31.94 | | |
| 24 | 4-chlorobenzylamino | methyl | C = 57.04; H = 4.42 | | 274.1 |
| | | | N = 25.59; Cl = 12.95 | | 276.1 |

a) solution: MeOH p.a. + HCOOH
b) solution: MeOH p.a. + H₂O +NH₃

TABLE 2
Compounds Prepared by the Method of Examples 8-18

| PYRAZOLO[4,3 -d]PYRIMIDINE | | | CHN | MS | |
|---|---|---|---|---|---|
| SUBSTITUENT | | | ANALYSES | ANALYSES-ZMD | |
| | C7 | C3 | [%] | [M − H]⁻ a) | [M + H]⁺ b) |
| 25 | 3-chloroanilino | ethyl | C = 57.04; H = 4.42 | | 274.1 |
| | | | N = 25.59; Cl = 12.95 | | 276.1 |
| 26 | anilino | ethyl | C = 65.26; H = 5.48 | | 240.1 |
| | | | N = 29.27 | | |
| 27 | 4-bromoanlino | ethyl | C = 49.07; H = 3.80 | 317.0 | 318.0 |
| | | | N = 22.01; Br = 25.11 | 319.0 | 320.0 |
| 28 | 4-chloroanilino | ethyl | C = 57.04; H = 4.42 | | 274.1 |
| | | | N = 25.59; Cl = 12.95 | | 276.1 |
| 29 | 2-hydroxybenzylamino | ethyl | C = 62.44; H = 5.61 | 268.1 | 270.1 |
| | | | N = 26.00 | | |
| 30 | 3-hydroxybenzylamino | ethyl | C = 62.44; H = 5.61 | 268.1 | 270.1 |
| | | | N = 26.00 | | |
| 31 | 4-hydroxybenzylamino | ethyl | C = 62.44; H = 5.61 | 268.1 | 270.1 |
| | | | N = 26.00 | | |
| 32 | 2-methylbenzylamino | ethyl | C = 67.39; H = 6.41 | | 268.2 |
| | | | N = 26.20 | | |
| 33 | 3-methylbenzylamino | ethyl | C = 67.39; H = 6.41 | | 268.2 |
| | | | N = 26.20 | | |
| 34 | 4-methoxybenzylamino | ethyl | C = 63.59; H = 6.05 | | 284.0 |
| | | | N = 24.72 | | |
| 35 | 2-chlorobenzylamino | ethyl | C = 58.44; H = 4.90 | | 288.1 |
| | | | N = 24.34; Cl = 12.32 | | 290.1 |
| 36 | 2-bromobenzylamino | ethyl | C = 50.62; H = 4.25 | | 333.0 |
| | | | N = 21.08; Br = 24.05 | | 335.0 |
| 37 | 3-chlorobenzylamino | ethyl | C = 58.44; H = 4.90 | | 288.1 |
| | | | N = 24.34; Cl = l2.32 | | 290.1 |
| 38 | 3-hydroxy-4-methoxybenzylamino | ethyl | C = 60.19; H = 5.72 | | 300.0 |
| | | | N = 23.40 | | |
| 39 | furfurylamino | ethyl | C = 59.25; H = 5.39 | | 244.1 |
| | | | N = 28.79 | | |
| 40 | allylamino | ethyl | C = 59.10; H = 6.45 | | 204.1 |
| | | | N = 34.46 | | |
| 41 | cyclohexylamino | ethyl | C = 63.65; H = 7.81 | | 246.2 |
| | | | N = 28.55 | | |
| 42 | 1,4-(trans)-cyclohexyldiamino | ethyl | C = 59.98; H = 7.74 | | 261.0 |
| | | | N = 32.28 | | |
| 43 | 1,2-(cis)-cyclohexyldiamino | ethyl | C = 59.98; H = 7.74 | | 261.0 |
| | | | N = 32.28 | | |
| 44 | cyclopentylamino | ethyl | C = 62.31; H = 7.41 | | 232.2 |
| | | | N = 30.28 | | |
| 45 | cyclobutylamino | ethyl | C = 60.81; H = 6.96 | | 218.1 |
| | | | N = 32.23 | | |
| 46 | (isopent-2-en-1-yl)amino | ethyl | C = 62.3; H = 7.41 | | 232.2 |
| | | | N = 30.28 | | |
| 47 | pentylamino | ethyl | C = 61.78; H = 8.21 | | 234.0 |
| | | | N = 30.02 | | |
| 48 | 4-chlorobenzylamino | ethyl | C = 58.44; H = 4.90 | | 288.1 |
| | | | N = 24.34; Cl = 12.32 | | 290.1 |

a) solution: MeOH p.a. + HCOOH
b) solution: MeOH p.a. + H₂O + NH₃

TABLE 3
Compounds Prepared by the Method of Examples 8-18

| PYRAZOLO[4,3-d]PYRIMIDINE | | | CHN | MS | |
|---|---|---|---|---|---|
| SUBSTITUENT | | | ANALYSES | ANALYSES-ZMD | |
| | C7 | C3 | [%] | [M − H]⁻ a) | [M + H]⁺ b) |
| 49 | 3-chloroanilino | isopropyl | C = 58.44; H = 4.90 | | 288.5 |
| | | | N = 24.34; Cl = 12.32 | | 290.5 |
| 50 | anilino | isopropyl | C = 66.38; H = 5.97 | | 254.1 |
| | | | N = 27.65 | | |
| 51 | 4-bronioanilino | isopropyl | C = 50.62; H = 4.25 | 331.1 | 332.1 |
| | | | N = 21.08; Br = 24.05 | 333.1 | 334.1 |
| 52 | 4-chloroanilino | isopropyl | C = 58.44; H = 4.90 | 286.1 | 288.1 |
| | | | N = 24.34; Cl = 12.32 | 288.1 | 290.1 |
| 53 | 2-hydroxybenzylamino | isopropyl | C = 63.59; H = 6.05 | 272.3 | 274.3 |
| | | | N = 24.72 | | |
| 54 | 3-hydroxybenzylamino | isopropyl | C = 63.59; H = 6.05 | 272.3 | 274.3 |
| | | | N = 24.72 | | |
| 55 | 4-hydroxybenzylamino | isopropyl | C = 63.59; H = 6.05 | 272.3 | 274.3 |
| | | | N = 24.72 | | |
| 56 | 2-methylbenzylamino | isopropyl | C = 68.30; H = 6.81 | | 282.2 |
| | | | N = 24.89 | | |
| 57 | 3-methylbenzylamino | isopropyl | C = 68.30; H = 6.81 | | 282.2 |
| | | | N = 24.89 | | |
| 58 | 4-methoxybenzylamino | isopropyl | C = 64.63; H = 6.44 | 296.3 | 298.3 |
| | | | N = 23.55 | | |
| 59 | 2-chlorobenzylamino | isopropyl | C = 59.70; H = 5.34 | | 302.1 |
| | | | N = 23.21; Cl = 11.75 | | 304.1 |
| 60 | 2-bromobenzylamino | isopropyl | C = 52.04; H = 4.66 | 344.1 | 346.1 |
| | | | N = 20.23; Cl = 23.08 | 346.1 | 348.1 |
| 61 | 3-chlorobenzylamino | isopropyl | C = 59.70; H = 5.34 | | 302.1 |
| | | | N = 23.21; Cl = 11.75 | | 304.1 |
| 62 | 3-hydroxy-4-methoxybenzylamino | isopropyl | C = 61.33; H = 6.11 | 312.3 | 314.3 |
| | | | N = 22.35 | | |
| 63 | furfurylamino | isopropyl | C = 60.69; H = 5.88 | | 258.3 |
| | | | N = 27.22 | | |
| 64 | allylamino | isopropyl | C = 60.81; H = 6.96 | | 218.1 |
| | | | N = 32.23 | | |
| 65 | cyclohexylamino | isopropyl | C = 64.84; H = 8.16 | | 260.2 |
| | | | N = 27.00 | | |
| 66 | 1,4-(trans)-cyclohexyldiamino | isopropyl | C = 61.29; H = 8.08 | 273.3 | 275.3 |
| | | | N = 30.63 | | |
| 67 | 1,2-(cis)-cyclohexyldiamino | isopropyl | C = 61.29; H = 8.08 | 273.3 | 275.3 |
| | | | N = 30.63 | | |
| 68 | cyclopentylamino | isopropyl | C = 63.65; H = 7.81 | | 246.2 |
| | | | N = 28.55 | | |
| 69 | cyclobutylamino | isopropyl | C = 62.31; H = 7.41 | | 232.2 |
| | | | N = 30.28 | | |
| 70 | isopent-(2-en)-1-ylamino | isopropyl | C = 63.65; H = 7.81 | | 246.5 |
| | | | N = 28.55 | | |
| 71 | pentylamino | isopropyl | C = 63.13; H = 8.56 | | 248.2 |
| | | | N = 28.31 | | |
| 72 | 4-chlorobenzylamino | isopropyl | C = 59.7O; H = 5.34 | | 302.1 |
| | | | N = 23.21; Cl 11.75 | | 304.1 |

a) solution: MeOH p.a. + HCOOH
b) solution: MeOH p.a. + H₂O + NH₃

### Example 19

### CDK Inhibition Assays

Proteins

Cyclin-dependent kinases (p34^{cdc2}, p33^{cdk2}) and cyclins (cyclin B, E) are produced in Sf9 insect cells coinfected with appropriate baculoviral constructs. The cells are harvested 68-72 hrs post infection in lysis buffer for 30 min on ice and the soluble fraction is recovered by centrifugation at 14.000 g for 10 min. The protein extract is stored at −80° C.

Lysis buffer: 50mM Tris pH 7.4, 150mM NaCl, 5 mM EDTA, 20 mM NaF, 1% Tween, 1 mM DTT, 0.1 mM PMSF, leupeptine, aprotonine.

Enzyme Inhibition Assays

To carry out experiments on kinetics under linear conditions, the final point test system for kinase activity measurement is used. The kinase is added to reaction mixture in such a way as to obtain linear activity with respect to the concentration of enzyme and with respect to time.

The p34^{cdc2} and p33^{cdk2} kinase inhibition determination involves the use of 1 mg/ml histone H1 (Sigma, type III-S) in the presence of 15 μM [γ-³³P]ATP (500-100 cpm/pmol) (ICN) in a final volume of 10 μl. Kinase activity is determined at 30° C. in the kinase buffer.

Tested compounds are usually dissolved to 100 mM solutions in DMSO, final concentration of DMSO in reaction mixture never exceeds 1%. The controls contain suitable dilutions of DMSO.

After 10 min, addition 3×SDS sample buffer stops the incubations. Phosphorylated proteins are separated electrophoretically using 10% SDS polyacrylamide gel. The measurement of kinase activity is done using digital image analysis.

The kinase activity is expressed as a percentage of maximum activity, the apparent inhibition constants are determined by graphic analysis.

Kinase buffer: 50 mM Hepes pH 7.4, 10 mM MgCl₂, 5 mM EGTA, 10 mM

2-glycerolphosphate, 1 mM NaF, 1 mM DTT

TABLE 4
Kinase Inhibitory Activity of Selected 3,7-Disubstituted Pyrazolo
[4,3-d]pyrimidine Derivatives

| SUBSTITUENT | | CDK1 | CDK2 |
|---|---|---|---|
| C7 | C3 | IC₅₀(μM) | IC₅₀(μM) |

| | | | |
|---|---|---|---|
| Olomoucine | | 7 | 7 |
| 3-chloroanlino | methyl | 14 | 16 |
| anilino | methyl | 22 | 21 |
| 3-chloro-5-aminoanilino | methyl | 19 | 24 |
| 3-chloro-4-carboxyanilino | methyl | 25 | 28 |
| 3-carboxy-4-chloroanilino | methyl | 24 | 29 |
| 3-carboxy-4-hydroxyanilino | methyl | 34 | 40 |
| 4-bromoanilino | methyl | 16 | 18 |
| 4-chloroanilino | methyl | 26 | 28 |
| 3-amino-4-chloroanilino | methyl | 27 | 28 |
| 3-chloro-4-aminoanilino | methyl | 26 | 28 |
| 2-hydroxybenzylamino | methyl | 4 | 5.2 |
| 3-hydroxybenzylamino | methyl | 5.5 | 7.2 |
| 2-methylbenzylamino | methyl | 18 | 17 |
| 3-methylbenzylamino | methyl | 28 | 31 |
| 2-chlorobenzylainino | methyl | 25 | 24 |
| 3-chlorobenzylamino | methyl | 8.8 | 9.4 |
| furfurylamino | methyl | 18 | 16 |
| allylamino | methyl | 42 | 48 |
| cyclohexylamino | methyl | 34 | 32 |
| cyclopentylamino | methyl | 29 | 46 |
| cyclobutylamino | methyl | 35 | 28 |
| isopentenylamino | methyl | 45 | 44 |
| 4-chlorobenzylamino | methyl | 20 | 18 |
| benzylamino | ethyl | 12 | 11 |
| 3-chloroanilino | ethyl | 24 | 18 |
| anilino | ethyl | 14 | 20 |
| 3-chloro-5-aminoanilino | ethyl | 20 | 15.5 |
| 3-chloro-4-carboxyanilino | ethyl | 36 | 32 |
| 3-carboxy-4-chloroanilino | ethyl | 38 | 40 |
| 3-carboxy-4-hydroxyanilino | ethyl | 24 | 22 |
| 4-bromoanilino | ethyl | 22 | 18 |
| 4-chloroanilino | ethyl | 14 | 16 |
| 3-amino-4-chloroanilino | ethyl | 23 | 22 |
| 3-chloro-4-aminoanilino | ethyl | 35 | 28 |
| 2-hydroxybenzylamino | ethyl | 6.2 | 7 |
| 3-hydroxybenzylamino | ethyl | 6 | 3.2 |
| 2-methylbenzylamino | ethyl | 15 | 14 |
| 3-methylbenzylamino | ethyl | 18 | 17 |
| 2-chlorobenzylamino | ethyl | 12 | 12 |
| 3-chlorobenzylamino | ethyl | 9.7 | 10.2 |
| furfurylamino | ethyl | 8.2 | 5.3 |
| allylamino | ethyl | 25 | 29 |
| cyclohexylamino | ethyl | 39 | 42 |
| cyclopentylamino | ethyl | 32 | 32 |
| cyclobutylamino | ethyl | 27 | 25 |
| isopentenylamino | ethyl | 45 | 41 |
| 4-chlorobenzylamino | ethyl | 26 | 19 |
| benzylamino | isopropyl | 1.3 | 0.5 |
| 3-chloroanilino | isopropyl | 2.0 | 0.8 |
| anilino | isopropyl | 4.4 | 5.2 |
| 3-chloro-5-aminoanilino | isopropyl | 6.1 | 5.8 |
| 3-chloro-4-carboxyanilino | isopropyl | 2.5 | 2.8 |
| 3-cazboxy-4-chloroanilino | isopropyl | 2.8 | 2.9 |
| 3-carboxy-4-hydroxyanilino | isopropyl | 3.1 | 4.2 |
| 4-bromoanilino | isopropyl | 1.7 | 1.9 |
| 4-chloroanilino | isopropyl | 2.1 | 3.1 |
| 3-amino-4-chloroanilino | isopropyl | 2.9 | 3.0 |
| 3-chloro-4-aminoanilino | isopropyl | 2.9 | 3.0 |
| 2-hydroxybenzylamino | isopropyl | 0.4 | 0.27 |
| 3-hydroxybenzylamino | isopropyl | 1.1 | 0.9 |
| 4-hydroxybenzylamino | isopropyl | 1.8 | 0.2 |
| 4-methoxybenzylamino | isopropyl | 2.3 | 1.0 |
| 2-methylbenzylamino | isopropyl | 2 | 1.4 |
| 3-methylbenzylamino | isopropyl | 2.2 | 3.1 |
| 2-chlorobenzylamino | isopropyl | 2.1 | 2.0 |
| 3-chlorobenzylamino | isopropyl | 8.8 | 9.4 |
| 3-hydroxy-4-methoxy | isopropyl | 0.9 | 0.2 |
| 2-bromobenzylamino | isopropyl | 7 | 9 |
| furfurylamino | isopropyl | 1.8 | 0.8 |
| allylamino | isopropyl | 16 | 14 |
| cyclohexylamino | isopropyl | 80 | 95 |
| cyclopentylamino | isopropyl | 18 | 20 |
| (2-aminocyclohexyl)amino | isopropyl | >100 | >100 |
| (4-aminocyclohexyl)amino | isopropyl | 80 | 70 |
| pentylamino | isopropyl | 1.7 | 1.4 |
| isopentenylamino | isopropyl | 4.5 | 1.3 |
| 4-chlorobenzylamino | isopropyl | 1.8 | 1.1 |

Table 4 shows the results of inhibitory activity of novel compounds against CDK1 and CDK2 in comparison with the data on a prototype compound (trisubstituted purine olomoucine). Most of the 3,7-disubstituted pyrazolo[4,3-d]pyrimidine derivatives showed marked inhibitory activity in in vitro kinase assays

### Example 20

### CDK Inhibitory Activity on Plant Kinases and Antimitotic Effects

Protein extraction and purification of pant CDK by binding to p13^{suc1}-beads or immunopurification with an antibody specific to the cdc2a-MS protein was carried out as described previously (Bögre et al. 1997, Plant Physiol. 113, 1997, 841-852). The MMK1 protein kinase was purified with a specific antibody from Vicia faba extracts as described by Bögre et al. 1997a, Plant Cell 9, 75-83). Protein kinase activity was measured as described above in Example 19. The quantification of radioactivity incorporated into histone H1 or myelin basic protein was undertaken using Phosphoimager (original gel images shown on FIG. 1). IC₅₀ were calculated from dose-response curves. The drugs inhibited the activity of immunopurified Vicia faba and alfalfa Cdc2-kinase. An observed transient arrest at the G1/S and G2/M indicated that inhibition of the Cdc2-kinase had an effect on both transitions. In contrast to the regular bipolar spindle in untreated cell, in drug-treated metaphase cells abnormally short and dense kinetochore microtubule fibres were observed. These microtubules were randomly arranged in the vicinity of the kinetochores and connected the chromosomes. Thus the chromosomes were not aligned on the metaphase plate but were arranged in a circle, with kinetochores pointing inwards and chromosome arms pointing outwards. γ-tubulin, which plays a role in microtubule nucleation, also localised to the centre of the monopolar spindle. The observed abnormalities in mitosis, after inhibition of Cdc2-kinase by specific drugs suggest a role for this enzyme in regulating some of the steps leading to a bipolar spindle structure (FIGS. 2 and 3).

TABLE 5
Kinase Inhibitory Activity of Selected 3,7-Disubstituted Pyrazolo
[4,3-d]pyrimidine Derivatives

| SUBSTITUENT | | CDC2a | MMK1 |
|---|---|---|---|
| C7 | C3 | IC₅₀(μM) | IC₅₀(μM) |

| | | | |
|---|---|---|---|
| Olomoucine | | 8 | 15.4 |
| 3-chloroanilino | methyl | 10 | 9.6 |
| anilino | methyl | 16 | 14 |
| 3-chloro-5-aminoanilino | methyl | 21 | 24 |
| 3-chloro-4-carboxyanilino | methyl | 27 | 29 |
| 3-carboxy-4-chloroanilino | methyl | 25 | 30 |
| 3-carboxy-4-hydroxyanilino | methyl | 32 | 44 |
| 4-bromoanilino | methyl | 28 | 28 |
| 4-chloroanilino | methyl | 26 | 26 |
| 3-amino-4-chloroanilino | methyl | 35 | 34 |
| 3-chloro-4-aminnoanilino | methyl | 30 | 32 |
| 2-hydroxybenzylamino | methyl | 4.2 | 4.8 |
| 3-hydroxybenzylamino | methyl | 5.8 | 8.3 |
| 2-methylbenzylamino | methyl | 20 | 20 |
| 3-methylbenzylamino | methyl | 30 | 31 |
| 2-chlorobenzylamino | methyl | 25 | 25 |
| 3-chlorobenzylamino | methyl | 8.4 | 10 |
| furfurylamino | methyl | 18 | 16 |
| allylamino | methyl | 45 | 50 |
| Cyclohexylamino | methyl | 35 | 30 |
| Cyclopentylamino | methyl | 30 | 45 |
| Cyclobutylamino | methyl | 35 | 28 |
| Isopentenylamino | methyl | 45 | 44 |
| 4-chlorobenzylamino | methyl | 20 | 18 |
| benzylamino | ethyl | 14 | 16 |
| 3-chloroanilino | ethyl | 22 | 21 |
| anilino | ethyl | 19 | 24 |
| 3-chloro-5-aminoanilino | ethyl | 25 | 28 |
| 3-chloro-4-carboxyanilino | ethyl | 24 | 29 |
| 3-carboxy-4-chloroanilino | ethyl | 34 | 40 |
| 3-carboxy-4-hydroxyanilino | ethyl | 16 | 18 |
| 4-bromoanilino | ethyl | 26 | 28 |
| 4-chloroanilino | ethyl | 27 | 28 |
| 3-amino-4-chloroanilino | ethyl | 26 | 28 |
| 3-chloro-4-aminoanilino | ethyl | 4 | 5.2 |
| 2-hydroxybenzylamino | ethyl | 5.5 | 7.2 |
| 3-hydroxybenzylamino | ethyl | 18 | 17 |
| 2-methylbenzylamino | ethyl | 28 | 31 |
| 3-methylbenzylamino | ethyl | 25 | 24 |
| 2-chlorobenzylamino | ethyl | 8.8 | 9.4 |
| 3-chlorobenzylamino | ethyl | 18 | 16 |
| furfurylamino | ethyl | 42 | 48 |
| allylamino | ethyl | 34 | 32 |
| cyclohexylamino | ethyl | 29 | 46 |
| cyclopentylamino | ethyl | 35 | 28 |
| cyclobutylamino | ethyl | 45 | 44 |
| isopentenylamino | ethyl | 20 | 18 |
| 4-chlorobenzylamino | ethyl | 8 | 5.3 |
| benzylamino | isopropyl | 1.1 | 1.2 |
| 3-chloroanilino | isopropyl | 2.0 | 0.8 |
| anilino | isopropyl | 4.4 | 5.2 |
| 3-chloro-5-aminoanilino | isopropyl | 2.8 | 2.9 |
| 3-chloro-4-carboxyanilino | isopropyl | 3.2 | 4.1 |
| 3-carboxy-4-chloroanilino | isopropyl | 1.6 | 1.7 |
| 3-carboxy-4-hydroxyanilino | isopropyl | 2.2 | 3.6 |
| 4-bromoanilino | isopropyl | 3.2 | 3.7 |
| 4-chloroanilino | isopropyl | 3.1 | 3.0 |
| 3-amino-4-chloroanilino | isopropyl | 4.1 | 1.8 |
| 3-chloro-4-aminoanilino | isopropyl | 0.6 | 0.4 |
| 2-hydroxybenzylamino | isopropyl | 1.7 | 1.3 |
| 3-hydroxybenzylamino | isopropyl | 2.1 | 2.7 |
| 4-hydroxybenzylamino | isopropyl | 2.2 | 1.8 |
| 4-methoxybenzylamino | isopropyl | 2.4 | 3.1 |
| 2-methylbenzylamino | isopropyl | 1.9 | 2.2 |
| 3-methylbenzylamino | isopropyl | 7.4 | 8.4 |
| 2-chlorobenzylamino | isopropyl | 1.7 | 0.7 |
| 3-chlorobenzylamino | isopropyl | 2.0 | 1.4 |
| 2-bromobenzylamino | isopropyl | 5.5 | 12 |
| furfurylamino | isopropyl | 4.3 | 5.3 |
| allylamino | isopropyl | 13.1 | 15 |
| cyclohexylamino | isopropyl | 30 | 34 |
| cyclopentylamino | isopropyl | 20 | 22 |
| isopentenylamino | isopropyl | 14 | 15 |
| (2-aminocyclohexyl)amino | isopropyl | >100 | >100 |
| pentylamino | isopropyl | 3.2 | 4.2 |
| 4-chlorobenzylamino | isopropyl | 1.7 | 1.6 |

Table 5 shows the results of inhibitory activity of novel compounds against plant in comparison with the data on the prototype compounds (disubstituted purines olomoucine, roscovitine and purvalanol A). Most of the 3,7-disubstituted pyrazolo[4,3-d]pyrimidine derivatives showed marked inhibitory activity in in vitro plant kinase assays.

### Example 21

### In Vitro Cytotoxic Activity of Novel Compounds

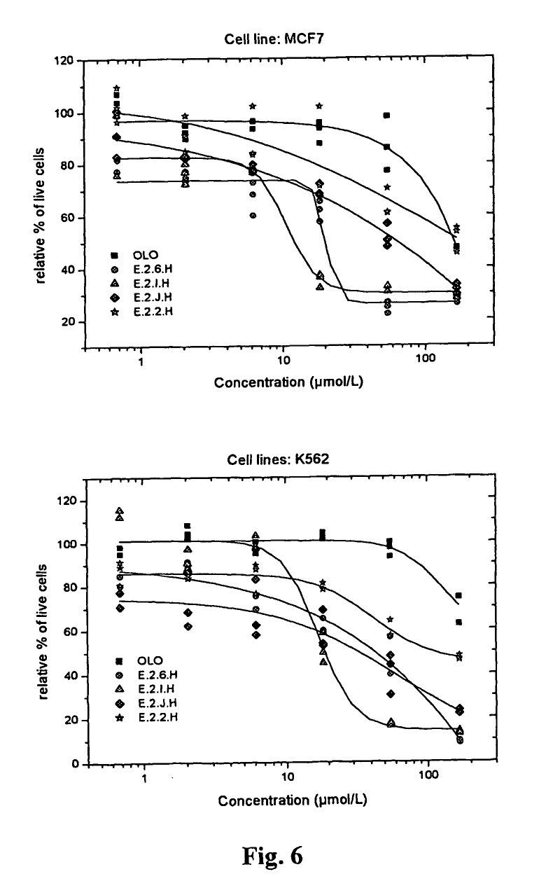

We have been using the following cell lines: HELA (human cervical carcinoma), MCF7 (human breast adenocarcinoma), NIH 3T3 (mouse fibroblasts), HOS (human osteogenic sarcoma), HL 60 (human promyelocytic leukemia), G 361 (human malignant melanoma), K562 (human chronic myeloblastic leukemia), CEM (human lymphoblastoid leukaemia). Tested drugs were added to the cell cultures in six different concentration and kept at 37° C. and 5% CO₂ for three days. All cell lines were grown in DMBM medium (Gibco BRL) supplemented with 10% (v/v) fetal bovine serum and L-glutamine and maintained at 37° C. in a humidified atmosphere with 5% CO₂. 10⁴ cells were seeded into each well of 96 well plate, allowed to stabilize for at least 2 h and then tested compounds were added at various concentrations ranging from 200 to 0.2 μM in triplicates. Three days after drug addition Calcein AM solution (Molecular Probes) was added and let to enter the cells for 1 hour. Fluorescence of viable cells was quantified employing Fluoroskan Ascent (Microsystems). The IC₅₀ value, the drug concentration lethal to 50% of the tumour cells, was calculated from the obtained dose response curves (FIG. 6).

Cytoxicity of novel compounds was tested on panel of cell lines of different histogenetic and species origin (Tab. 6). Higher activities were found in all tumour cell lines tested. Notably, the higher effectiveness of novel derivatives was also found in cell lines bearing various mutations or deletions in cell cycle associated proteins, e.g. HL-60, BT549, Hela, U2OS, MDA-MB231, and Saos2. It indicates that these substances should be equally effective in tumours with various alterations of tumour suppressor genes, namely p53, Rb, etc. Importantly, this observation distinguishes the novel compounds from flavopiridol and related compounds, as their biological activity is dependent on p53 status.

TABLE 6
Cytotoxicity of Novel Compounds for Different Cancer Cell Lines

| SUBSTITUENT | | MCF7 | K-562 |
|---|---|---|---|
| C7 | C3 | IC₅₀(μM) | IC₅₀(μM) |
| Olomoucine | | 131.8 | >167 |
| benzylamino | methyl | 67 | 29 |
| 3-chloroanilino | methyl | 24 | 35 |
| 2-hydroxybenzylamino | methyl | 67 | 55 |
| 3-hydroxybenzylamino | methyl | 119 | 143 |
| 4-hydroxybenzylamino | methyl | >167 | >167 |
| 3-hydroxy-4-methoxybenzylamino | methyl | 58 | 79 |
| 4-methoxybenzylamino | methyl | 35 | 53 |
| furfurylamino | methyl | 142 | >167 |
| pentylamino | methyl | 45 | 67 |
| cyclobutylamino | methyl | 89 | 101 |
| 4-aminocyclohexylamino | methyl | 75 | 115 |
| 2-bromobenzylamino | methyl | 56 | 68 |
| benzylamino | ethyl | 62 | 81 |
| 3-chloroanilino | ethyl | 19 | 28 |
| 2-hydroxybenzylamino | ethyl | 63 | 51 |
| 3-hydroxybenzylamino | ethyl | 111 | 132 |
| 3-hydroxy-4-methoxybenzylamino | ethyl | 52 | 72 |
| 4-methoxybenzylamino | ethyl | 31 | 48 |
| furfurylamino | ethyl | 135 | >167 |
| pentylamino | ethyl | 41 | 62 |
| cyclobutylamino | ethyl | 82 | 94 |
| 4-aminocyclohexylamino | ethyl | 71 | 110 |
| 2-bromobenzylamino | ethyl | 54 | 60 |
| benzylamino | isopropyl | 55 | 72 |
| 3-chloroanilino | isopropyl | 9 | 12 |
| anilino | isopropyl | 15 | 21 |
| 3-chloro-5-aminoanilino | isopropyl | 29 | 35 |
| 3-chloro-4-carboxyanilino | isopropyl | 46 | 69 |
| 3-carboxy-4-chloroanilino | isopropyl | 0.4 | 1 |
| 3-carboxy-4-hydroxyanilino | isopropyl | 12 | 25 |
| 4-bromoanilino | isopropyl | 5 | 7 |
| 4-chloroanilino | isopropyl | 3 | 4 |
| 3-amino-4-chloroanilino | isopropyl | 0.2 | 0.3 |
| 3-chloro-4-aminoanilino | isopropyl | 12 | 13 |
| 2-hydroxybenzylamino | isopropyl | 63 | 50 |
| 3-hydroxybenzylamino | isopropyl | 105 | 132 |
| 4-hydroxybenzylamino | isopropyl | 152 | >167 |
| 3-hydroxy-4-methoxybenzylamino | isopropyl | 45 | 68 |
| 4-methoxybenzylamino | isopropyl | 28 | 41 |
| 2-methylbenzylamino | isopropyl | 63 | 75 |
| 3-methylbenzylamino | isopropyl | 76 | 94 |
| 2-chlorobenzylamino | isopropyl | 15 | 21 |
| 3-chlorobenzylamino | isopropyl | 24 | 26 |
| furfurylamino | isopropyl | 130 | >167 |
| allylamino | isopropyl | 64 | 77 |
| cyclohexylamino | isopropyl | 95 | 98 |
| pentylamino | isopropyl | 32 | 61 |
| cyclobutylamino | isopropyl | 45 | 60 |
| isopentenylamino | isopropyl | >167 | >167 |
| 2-aminocyclohexylamino | isopropyl | >167 | >167 |
| 4-aminocyclohexylamino | isopropyl | 68 | 107 |
| 2-bromobenzylamino | isopropyl | 42 | 57 |
| 2-hydroxy-3-methoxybenzylamino | isopropyl | 0.6 | 0.8 |
| 2-hydroxy-4-methoxybenzylamino | isopropyl | 0.2 | 0.5 |
| 2-hydroxy-5-methoxybenzylamino | isopropyl | 0.9 | 1.2 |
| 2-aminobenzylamino | isopropyl | 0.8 | 2.1 |

### Example 9

### Novel Compounds Have Cytotoxic Effects for Plant Cells and Induce their Apoptosis

The novel compounds have also been tested in tobacco callus bioassay for cytotoxicity (herbicidal activity) and induction of cell death. The compounds to be tested were dissolved in dimethylsulfoxide (DMSO) and the solution brought up to 10⁻³ M with distilled water. This tock solution was further diluted in the respective media used for the tobacco bioassay to concentration ranging from 10⁻⁸ M to 10⁻⁴ M. The final concentration of DMSO in the media did not exceed 0.2%, and therefore did not affect biological activity in the assay system used. Cytokinin-dependent tobacco callus Nicotiana tabacum L. cv. Wisconsins 38 Murashige-Skoog medium, containing per 1 liter: 4 μmol nicotinic acid, 2.4 μmol pyridoxine hydrochloride, 1.2 μmol thiamine, 26.6 μmol glycine, 1.37 μmol glutamine, 1.8 μmol myoinositol, 30 g of sucrose, 8 g of agar, 5.37 μmol α-naphtylacetic acid and 0.5 μmol 6-benzylaminopurine. Subcultivation was carried out every three weeks. Fourteen days before the bioassay, the callus tissue was transferred to the media without 6-benzylaminopurine. Compounds were tested with two different concentrations of 6-benzylaminopurine (10⁻⁵ M and 10⁻⁶ M). Inhibitory growth activity was determined from the increase in fresh callus weight after four weeks of cultivation. Five replicates were prepared for each concentration tested and the entire test was repeated at least twice. Inhibitory activity was compared with growth response curve of 6-benzylaminopurine in the range from 10⁻⁸ M to 10⁻⁴ M and IC50 was calculated for each compound for 10⁻⁵ M and 10⁻⁶ M of 6-benzylaminopurine.

TABLE 7
Cytotoxicity of Novel Compounds for Tobacco Plant Cells
Cultivated in vitro

| SUBSTITUENT | | 10⁻⁵ M BAP | 10⁻⁶ M BAP |
|---|---|---|---|
| C7 | C3 | IC₅₀(μM) | IC₅₀(μM) |

| | | | |
|---|---|---|---|
| OLOMOUCIN | | >50 | >50 |
| benzylamino | isopropyl | 24 | 6.5 |
| 3-chloroanilino | isopropyl | 34 | 8.2 |
| anilino | isopropyl | 41 | 5.3 |
| 2-methylbenzylamino | isopropyl | 14 | 4.2 |
| 3-methylbenzylamino | isopropyl | 26 | 8.5 |
| 2-chlorobenzylamino | isopropyl | 31 | 4.6 |
| 3-chlorobenzylamino | isopropyl | 20 | 1.7 |
| furfurylamino | isopropyl | 25 | 9.4 |
| allylamino | isopropyl | 18 | 3.7 |
| cyclohexylamino | isopropyl | 46 | 9.4 |
| cyclopentylamino | isopropyl | 15.6 | 8.4 |
| cyctobutylamino | isopropyt | 12.1 | 4.3 |
| isopentenylamino | isopropyl | 15.3 | 1.7 |
| 4-chlorobenzylamino | isopropyl | 12.1 | 4.7 |

Table 7 shows the results of inhibitory activity of novel compounds on growth of tobacco cells cultivated in vitro in comparison with the data on the prototype compound (trisubstituted purine olomoucine). Most of the 3,7-disubstituted pyrazolo[4,3-d]pyrimidine derivatives showed marked inhibitory activity on in vitro growth. Furthermore, these compounds are able to induce apoptosis in plants cells (are able to kill plant cells) and induce strong antimitotic activities (see FIGS. 4 and 5). A dose-dependent inhibition of the cell cycle in G1/S and G2/M transition points was observed. The appearance of DNA fragmentation observed by DNA double strand breaks labelling in situ started 3 h after drugs addition with highest frequency after 24-48 h of treatment, when oligonucleosomal DNA ladder occurred. The high doses of roscovitine , bohemine which induced apoptosis were shown to downregulate in vivo activity of cdk; decrease of cdk protein level was shown by Western blotting and immunofluorescence labelling. Microtubule reorganization contributing to apoptotic morphology was observed. The results presented here clearly show that the novel compounds exhibit herbicidal activity.

1. 3-,7-disubstituted pyrazolo[4,3-d]pyrimidines represented by the general formula I 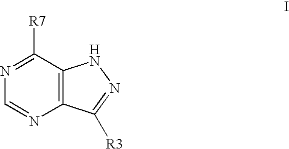
and pharmaceutically acceptable salts thereof, wherein
R3 is selected from the group consisting of
alkyl, cycloalkyl, cycloalkyl alkyl, cycloheteroalkyl alkyl, cycloheteroalkyl, aryl, heterocycle, heteroaryl, arylalkyl, heteroarylalkyl, and heteroalkyl, wherein each of the groups may optionally be substituted,
R7 is selected from the group consisting of halogen, hydroxyl, hydroxylamino, amino, carboxyl, cyano, nitro, amido, sulfo, sulfamido, carbamino, unsubstituted or substituted alkyl, substituted or unsubstituted cycloalkyl, substituted or unsubstituted arylalkyl, substituted or unsubstituted heteroalkyl, substituted or unsubstituted heteroarylalkyl, substituted or unsubstituted cycloalkyl alkyl, substituted or unsubstituted cycloheteroalkyl alkyl;
R7′-X— wherein X is an —NH—, —N(alkyl)-, —O— or —S— moiety and
R7′ is selected from the group consisting of H, alkyl, cycloalkyl, aryl, alkylcycloalkyl, arylalkyl, heterocycle, heterocycloalkyl, substituted alkyl, substituted cycloalkyl, substituted aryl, substituted arylalkyl, substituted heterocycle, substituted heteroaryl, substituted heteroarylalkyl, substituted heteroalkyl, substituted cycloalkyl alkyl and substituted cycloheteroalkyl alkyl.

2. 3-,7-disubstituted pyrazolo[4,3-d]pyrimidines according to claim 1, wherein the groups are substituted by more than one halogen, hydroxyl, amino, mercapto, carboxyl, cyano, nitro, amido, sulfo, sulfamido, carbamino, alkyl, alkoxy, and/or substituted alkyl group.

3. 3-,7-disubstituted pyrazolo[4,3-d]pyrimidines according to claim 1, wherein
R3 is selected from the group consisting of
alkyl, cycloalkyl, cycloalkyl alkyl, cycloheteroalkyl, cycloheteroalkylalkyl, cycloalkyl alkyl, aryl, arylalkyl, heteroaryl, heteroarylalkyl, wherein each of the groups may be optionally be substituted by 1-3 halogens like fluoro and chloro;
R7 is selected from the group consisting of halogen, hydroxyl, hydroxylamino, amino, hydrazino, carboxyl, cyano, nitro, amido, sulfo, sulfamido, carbamino, NHCONH2, NHC(═NH)NH2, alkyl, cycloalkyl, cycloalkylalkyl, aryl, arylalkyl, heteroalkyl, heteroaryl, heteroarylalkyl, cycloheteroalkyl, cycloheteroalkylalkyl, which is substituted independently at each occurrence with 0-5 substituents selected from the group halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
R7′-X wherein X is —NH—, —O—, —S—; or —N(alkyl)- and
R7′-X, wherein X is preferably —N(alkyl)- selected at each occurrence from the group methyl, ethyl, propyl, isopropyl, ethinyl, allyl, propargyl, isopent.2-en-1-yl;
R7′ is
C₁-C₈ branched or unbranched alkyl, alkenyl or alkinyl selected from the group consisting of methyl, ethyl, isopropyl, butyl, isobutyl, allyl, propargyl, isopent-2en-1-yl, and 2-methylallyl, which are substituted independently at each occurrence with 0-5 substituents selected from the group consisting of halogen, hydroxy, alkoxy, amino, hydrazo, mercapto, carboxyl, cyano, nitro, amido, sulfo, sulfamido, acylamino, acyloxy, alkylamino, dialkylamino, alkylthio and carbamoyl group;
acyl, —C(O)Rₐ, wherein Rₐ is C₁-C₆ branched or unbranched alkyl, alkenyl or alkinyl selected from the group consisting of methyl, ethyl, isopropyl, butyl, isobutyl, allyl, propargyl, isopent-2en-1-yl, and 2-methylallyl, which are substituted independently at each occurrence with 0-5 substituents selected from the group consisting of halogen, hydroxyl, alkoxy, amino, hydrazo, mercapto, carboxyl, cyano, nitro, amido, sulfo, sulfamido, acylamino, acyloxy, alkylamino, dialkylamino, alkylthio and carbamoyl group;
amido, —C(O)NR_{b}R_{c}, wherein R_{b} and R_{c} is C₁-C₆ branched or unbranched alkyl, alkenyl or alkinyl selected from the group defined above for C₁-C₆ branched or unbranched alkyl, which is substituted independently at each occurrence with 0-5 substituents selected from the group defined above for acyl;
sulfo, —SO₃R_{d}, wherein R_{d} is C₁-C₆ branched or unbranched alkyl, alkenyl or alkinyl selected from the group consisting of methyl, ethyl, isopropyl, butyl, isobutyl, allyl, propargyl, isopent-2en-1-yl, and 2-methylallyl, which is substituted independently at each occurrence with 0-5 substituents selected from the group defined above for acyl;
C₃-C₁₅ cycloalkyl selected from the group consisting of cyclopropyl, cyclobutyl, cyclopentyl, cyclohexyl, cycloheptyl or adamantyl;
substituted C₃-C₁₅ cycloalkyl selected from the group consisting of cyclopropyl, cyclobutyl, cyclopentyl, cyclohexyl, cycloheptyl or adamantyl substituted independently at each occurrence with 0-5 substituents selected from the group defined above for acyl;
R_{f} (cycloalkyl), wherein R_{f} is
C₁-C₆ alkyl, alkenyl or alkinyl group defined above
C₃-C₁₅ cycloalkyl is selected from the group consisting of cyclopropyl, cyclobutyl, cyclopentyl, cyclohexyl, cycloheptyl or adamantyl;
substituted C₃-C₁₅ cycloalkyl selected from the group consisting of cyclopropyl, cyclobutyl, cyclopentyl, cyclohexyl, cycloheptyl or adamantyl substituted independently at each occurrence with 0-5 substituents selected from the group defined above for acyl;
aryl is selected from the group consisting of phenyl, biphenyl, naphthyl, tetrahydronaphtyl, fluorenyl, indenyl or fenanthrenyl substituted independently at each occurrence with 0-5 substituents selected from the group defined above for acyl;
heterocycle is selected from the group consisting of thienyl, furyl, pyranyl, pyrrolyl, imidazolyl, pyrazolyl, pyridyl, pyrazinyl, pyrimidinyl, pyridazinyl, isothiazolyl, isoxazyl substituted independently at each occurrence with 0-5 substituents selected from the group defined above for acyl;
heteroalkyl is —R_{g}-Het, wherein
R_{g} is C₁-C₆ alkyl, alkenyl or alkinyl selected from the group methylen, 1,2-ethyliden, 1,3-propiliden, 1,4-butyliden, pentamethylen, hexamethylen, ethylendiyl, allyl-1,3-diyl, methylethan-1,1-diyl, methylethan-1,2-diyl, butan-1,3-diyl, which is substituted independently at each occurrence with 0-5 substituents selected from the group halogen, hydroxy, alkoxy, cyano;
Het is heterocycle as defined above;
heteroaryl is —Rₕ-HetAr, wherein
Rₕ is C₁-C₆ alkyl, alkenyl or alkinyl selected from the group above, and HetAr is selected from the group consisting of benzothienyl, naphthothienyl, benzofuranyl, chromenyl, indolyl, isoindolyl, indazolyl, qinolyl, isoqinolyl, ftalazinyl, qinaxalinyl, cinnolinyl, qinazolinyl substituted independently at each occurrence with 0-5 substituents selected from the group defined above for acyl;
arylalkyl is —RᵢAr, wherein
Rᵢ is C₁-C₆ alkyl, alkenyl or alkinyl defined above and
Ar is aryl as defined above;
cycloheteroalkyl is selected from the group consisting of piperidinyl, piperazinyl, morfolinyl, pyrrolidinyl, imidazolidinyl substituted independently at each occurrence with 0-5 substituents selected from the group defined above for acyl; cycloheteroalkyl alkyl, -
Rⱼ (cycloheteroalkyl), wherein
Rⱼ is arylalkyl —RᵢAr, wherein
Rᵢ is C₁-C₆ alkyl, alkenyl or alkinyl defined above, and
Ar is aryl as defined above, and
cycloheteroalkyl is selected from the group consisting of piperidinyl, piperazinyl, morfolinyl, pyrrolidinyl, imidazolidinyl substituted independently at each occurrence with 0-5 substituents selected from the group defined above for acyl;
heteroarylalkyl is 'Rₖ-HetAr, wherein
Rₖ is C₁-C₆ alkyl, alkenyl or alkinyl as defined above, and
HetAr is heteroaryl group as defined above.

4. 3-,7-disubstituted pyrazolo[4,3-d]pyrimidines according to claim 1, which has independently at each occurrence (R) or (S) configuration in R3 or R7 in case of their chirality.

5. A method of preparing the 3-,7-disubstituted pyrazolo[4,3-d]pyrimidine of the formula I, according to claim 1 wherein R7 and R3 substituents are as defined in claim 1, wherein 3-Substituted-7-hydroxypyrazolo[4,3-d]pyrimidines are chlorinated, preferably with SOCl₂ or POCl₃ or PCl₅ or, or 7-hydroxy group is displaced by mercapto group, preferably by action of P₂S₅,
the thus obtained 7-chloro-3-substituted pyrazolo[4,3-d]pyrimidines are then reacted with an appropriate reactant nucleophil, or 7-mercapto-3-substituted pyrazolo[4,3-d]pyrimidines are reacted with an alkylating agent, and the resulting 3,7-disubstituted pyrazolo[4,3-d]pyrimidine is optionally isolated.

6. The method of claim 5, wherein the appropriate nucleophil R7′-X—Y is selected from the group consisting of 1) amine (ammonium hydroxide, hydrazine, hydroxylamine, benzylamine; 2-,3-,4-hydroxybenzylamine; dihydroxybenzylamine; and 3-chloroaniline, or 2) lithium, natrium, and kalium salt of alkohole or mercaptane.

7. The method of claim 5, wherein the alkylating agent is selected from the group consisting of alkylhalogenide, alkyltoluensulfonate, and alkylmesylate.

8. The method of claim 5, wherein the resulting 3,7-disubstituted pyrazolo[4,3-d]pyrimidine is isolated by chromatography on silica gel followed by crystallization or only by crystallization.

9. A method for inhibiting cell proliferation in mammals comprising administering effective amount of a compound according to claim 1 or a pharmaceutically acceptable salt of such a compound together with a pharmaceutical carrier.

10. A method of treating cancer, or psoriasis, rheumatoid arthritis, lupus, type I diabetes, multiple sclerosis, restenosis, polycystic kidney disease, graft rejection, graft versus host disease and gout, parasitoses such as those caused by fungi or protists, or Alzheimer's disease, or as antineurogenerative drugs, or to suppress immunostimulation comprising administering effective amount of 3-,7-disubstituted pyrazolo[4,3-d]pyrimidines according to claim 1 or a pharmaceutically acceptable salt thereof together with a pharmaceutical carrier.

11. A method of treating cancer comprising administering effective amount 3-,7-disubstituted pyrazolo[4,3-d]pyrimidines according to claim 1 or a pharmaceutically acceptable salt thereof in combination with usually used cytostatics, such as mitoxantrone, cis-platinum, methotrexate, taxol, or doxorubicin.

12. A method of treating an hyperproliferative skin disease in a human suffering therefrom by actinic keratosis, Bowen's disease, papilloma, seborrheic keratosis, toxic eczema, atopic dermatitis and ichthyosis comprising administering to a subject a therapeutically effective amount of 3-,7-disubstituted pyrazolo[4,3-d]pyrimidines according to claim 1 or a pharmaceutically acceptable salt thereof together with a pharmaceutical carrier.

13. A method of treating viral infections comprising administering to a subject a therapeutically effective amount of 3-,7-disubstituted pyrazolo[4,3-d]pyrimidines according to claim 1 or a pharmaceutically acceptable salt thereof together with a pharmaceutical carrier.

14. 3-,7-disubstituted pyrazolo[4,3-d]pyrimidines according to claim 1 as a therapeutic agent.

15. Use of 3-,7-disubstituted pyrazolo[4,3-d]pyrimidines according to claim 1 in the preparation of a medicament for the treatment of cancer, or psoriasis, rheumatoid arthritis, lupus, type I diabetes, multiple sclerosis, restenosis, polycystic kidney disease, graft rejection, graft versus host disease and gout, parasitoses such as those caused by fungi or protists, or Alzheimer's disease, asthma, actinic keratosis, Bowen's disease, papilloma, seborrheic keratosis, toxic eczema, atopic dermatitis and ichthyosis, cardiovascular, neurodegenerative, viral and inflammatory diseases.
